SEQUENCE LISTING (34 sequences)

SEQ ID NO: 1 - DNA, 11337 bp, Homo sapiens
  CDS (88)..(8781)
cggaaatgatccaataatatctactatagagaagctgaatatactccatttggagtgaacagcccggattgttacagaattttcaagatgacagatccaatgatggacttttttgatgatgccaatctttttggtgagaccttagaaggtttgtcagatgatgcatttgtacaaccaggacctgtttcactagttgatgaattgaatttgggtgcagaatttgaaccgttgcacatagattcactgaaccatgttcaaggtactccaacacatcagaagatgactgattttgaacagttaaatcaatttgattcaataaaatttcaccatgttaatcaatcttttggtagcccagctgaacatgtgttatctccacactctcagtttaattgttctccaatccatccccaaaaccaacccaatggtttgtttccagatgtatcagatggcagtccaatgtggggccatcagacagctactaccatttcaaatcaaaatggatctccttttcaccaacaaggacattcacactctatgcatcaaaataaaagctttgtggcacaccatgactttgccttatttcaggccaatgaacaacaaacacagtgtacttcactacgctcacaacaaaacagaaataatctcaacccagggcagaattctcttagccagtctaaaaattttatgaatgtttctggtccacatagagtcaatgttaaccacccaccacagatgactaatgcatctaattcacaacagtctatttcaatgcagcaattttctcaaacgtcaaatccttcagcacacttccacaagtgtagcagtcatcaagaaggaaattttaatggaccttccccaaatatgacttcttgttctgtcagtaattcacagcaattttcttcacattattccttttccagtaatcatatatcaccaaacagtctacttcagtcctctgcagttcttgcatctaatcatacaaatcagactttatctgattttactggaagtaattccttttcacctcatagaggaatcaagcaagaatctactcagcatatcctaaaccccaatacatcattgaattcaaataatttccaaatattgcattcatcacatcctcagggtaattatagcaattcaaaattatctcctgtgcacatgaacttcccagatcctgttgactcaggaactcaaatgggccatttcaatgatcatgtagaaactaatggcttttcatctttagaagagaatttacttcatcaagtggaatctcaaactgagccattcacaggacttgaccccgaggacctccttcaggagggtcttcttcctcactttgatgagtcaacattcggacaagataattcaagtcacattttagatcatgaccttgatcggcagtttacttcgcatctggtaacacggccttctgatatggctcagactcagttgcaaagtcaggctcggagttggcattcatcattttctaatcatcagcatttacatgacagaaatcacctatgtttacagcgacagcctccatcttccaagaagagcgatggttctgggacatatactaagttgcagaatacccaggtgagggtcatgtctgagaagaagcagagaaaaaaggtggaatcagaaagcaagcaagaaaaggctaatcgtataatatcagaggccatagcaaaagcaaaggagcgtggggaacgcaatattccacgagtaatgagccctgaaaactttcctactgcttcagttgaaggaaaagaggaaaagaaaggtagaaggatgaaatccaagccaaaggacaaagacagcaaaaaaacaaaaacatgttctaagttaaaagagaagacaaaaattggcaaactcattattacattgggtaagaaacaaaaaagaaagaatgagtcttcagatgaaatatctgatgcagaacagatgccacagcatacattaaaagatcaagactctcaaaaaagaagatcaaatcgacaaattaaaagaaaaaaatacgcagaagatatagaagggaagcaatctgaagaagaggttaaaggttctatgaaaataaaaaagaattcagctcctttacctggtgaacagcctttacaattgtttgtggagaatccgagtgaagaagatgctgcaattgtagacaaaattctatcttctagaaccgtaaaaaaggaaatatcacctggagtgatgattgatacagaagaatttttcgtaaaatacaagaattactcctatcttcactgtgagtgggccacagaagagcagcttttgaaagataaaaggatccagcagaaaatcaaacgattcaaattgagacaagcacaaagagcacatttttttgcagacatggaagaagaaccatttaacccagactacgttgaagtagacagagtattagaagtctctttttgtgaagataaggatactggtgagcctgttatttactacttagtaaaatggtgctcattgccatatgaagatagtacttgggaactaaaagaagatgtagatcttgcaaaaatagaagagtttgaacaactgcaagcttcaaggcctgacacaagacgtttggaccgtcctccttctaatatttggaagaaaatagatcaatccagggactataaaaatggcaatcaactcagggaatatcaactggaaggactcaactggctcttgttcaattggtacaatagacgaaactgcatcttagcagatgaaatgggtcttggcaaaactattcaatcaattacattcctctatgaaatccttctgactggtataagaggacctttcctgattattgctccactttctactattgcaaactgggagagagaatttcgtacgtggactgatattaacgttgtggtttatcatgggagcctgattagcagacaaatgatacagcaatacgagatgtacttcagggattcacaggggcgtatcattcgaggagcttacagattccaagccatcatcaccacttttgaaatgattcttggaggctgtggagagcttaatgcaattgaatggcgatgtgtgattattgatgaagcacataggttaaaaaataaaaattgtaaactcttagagggcctgaaactcatgaatctggaacacaaggtgcttttgactggcacccctctccaaaatacagttgaagaactatttagtcttcttcactttcttgaacccttaaggtttccttctgaatcaacatttatgcaagaatttggggatctgaaaacagaggaacaggtacagaaacttcaggctatcctgaaaccaatgatgttgagacgattaaaagaagatgtggaaaagaagttggcacctaaagaagaaaccatcattgaagtagaacttactaatattcaaaagaaatactaccgggctatcttggaaaagaacttttcttttttatccaaaggagcaggacaaactaatgtacctaacttggtcaataccatgatggagctcaggaaatgttgtaatcatccatatcttataaaaggtgctgaggagaaaatacttggagaatttagagatacttacaatccagctgcttctgattttcatcttcaagcaatgatccagtctgctggtaaattggtccttattgataaattgcttcccaaaatgaaagccggaggtcataaagtgctcatcttctctcaaatggttcgttgccttgacattctggaggactatctcatacataaaagatacttatatgagcgaattgatggcagagtcagaggaaatcttcggcaagctgctatagatagatttagtaaacctgattcagatagatttgtttttctcctgtgtacccgagctggtgggttgggcatcaacttaactgcagctgatacatgtataatttttgattctgattggaatcctcagaatgatcttcaggcccaagctcgttgccacagaattggtcagaacaaagcagttaaagtctacagactggtaactcgtaactcatatgagagagagatgtttgaccgagccagtttgaaactgggcctagataaagctgtgttacagagcatgagtggaagagaaagtaatgttggtggtattcagcagctttccaaaaaggaaatagaagatctgcttcgaagaggtgcttatggtgctattatggaggaagaagatgaaggctctaaattctgcgaagaggatatcgatcagattttactacgtcgtacaaaaactattacaattgaatcagaaggacgtgggtcaacatttgccaaggcgagttttgtggcatctggaaaccggacagatatttctttagatgatcccaacttctggcaaaaatgggctaaaaaggcagaaatagatatagaggccatcagtggcagaaacagcttggttattgacactccaagaattaggaagcaaacaagaccttttagtgccacaaaagatgaattggctgaattatctgaagctgaaagtgaaggagatgaaaagcccaaactccggagaccctgtgaccgttccaatggctatggaagaactgaatgctttagagttgagaaaaacctgctagtttatgggtggggccgatggagagagattctatctcatggccgtttcaaaaggcagctaaatgaacacgatgtagagataatttgccgagctctcttagcatattgccttgttcactaccgaggagatgagaagattaaaggtttcatatgggatctcattactccaactgaagatggacagacacgagagctacagaatcatctaggcctatcagctcctgtacccaggggtcgaaaagggaagaaagtaaaaactcaaacaagctcatttgatatacaaaaagcagaatggcttcgaaaatataatcccgagcagctccttcaagatgaaggctacaaaaaacatataaaacaccactgtaataaggttttgcttcgtgtgagaatgctgtattatctaaagcaagaagttattggaaatgagtgtcagaaagtatttgatggagttgatgcaagtgacattgatgtttgggtaccagaaccagaccactcagaagttcctgctgagtggtgggattttgatgctgataagtcactccttattggagtttttaaacatggatatgaaaaatataacactattcgagcagacccagcattatgcttcttggaaagagtgggaaaacctgatgagaaagcagttgctgctgaacagagagcgaatgattatatggatggggatgtggaagatccagaatacaaacctgccccagccatctttaaagatgatatagaggatgatgtttcctcaccaggagatcttgttatagcagatggagatggtcaactgatggagggtgataaagtatattggcctactcaatcagctttaaccacacgtttgaggcgtctcatcactgcataccagcgtactaataaaaacagacaaattcaacagatacaaccgactttctcggtgcctaccagtgtaatgcagcctatttatgaggaagccactcttaatcctaaaatggcagccaagatagaaagacagcaaagatggacaagaagagaagaagctgacttttatagggttgtatctacatttggagtggtttttgaccctgacagaggccaatttgattggacaaaatttagagctatggctaggctacataagaaaactgatgatagtttggaaaaatatttgtacgcattcatgtccatgtgtcggagggtttgtcgtcttccttccaaagaagaattggtggatccaaatatttttatccagcccatcacagaagaacgtgcttctaggactttgtatcgcattgaacttctaaggaaagtacgggaacaggcccttcgacatccacagttgtttgaacgcttgaagctttgccatccaaatccagatttaccagtctggtgggaatgtggccctcatgatagggatttgcttattggtgctgccaaacacggggtgagccgaacagactatcacattcttcgtgatcctgaactctcatttatggcagctcagaggaactacagtcaaagtaagatggctcattcaaggacttctaccccacttctacagcaatatcaagtagcactttctgcttctcctcttacctctctacctaggctcctagatgctaaaggtattattctagaggagatgaaagttaaaagtgaaaaccttaaagaggagcctcagtcttctgaagaagaatctatgtcttctgtggaaaccaggacactaataaaatctgagcctgtaagtccaaagaatggtgttttaccacaggctactggagaccagaaatctggtggaaaatgtgaaacagacagacgcatggttgcagccagaacagaacccctaactccaaacccagcttctaagaaaccaagagtccacaaaaggggatcagaatctagttctgattctgactcagattctgagagatcatcttgttcttccagatcatcttcttcctcatcatcctcttcttgctcccactctcgatcaggctctagttcttcttcatcttcatcttgttcttcagcatcttcttcatcctcttcctccacctcttcctcctcctcctcctcttcatcttcatcagaagaaagtgacagtgatgaagaagaagcccaaaaacgagaaagtactactcacatgaaagcctatgatgaagaaagcgtcgcgtcactgagcactacccaggatgagactcaggatagttttcagatgaacaatgggacaccagagtctgcttatatcttacaaggtggatatatgctggcagcctcgtattggccaaaggatcgtgtgatgatcaataggttggacagtatttgtcaaacagttctgaaaggaaagtggccttcagctagaagaagttatgatgctaacacagtggcttctttctataccacaaaactgctggacagccctggagcagctacagaatacagcgagcccagtgtacccactcccccaggtgccggtgttaaagaagaacatgatcagtcaacacagatgtcaaaggtgaagaagcatgtacgagaaaaggagtttacagtgaaaatcaaagacgaaggtggtttgaagttgacatttcagaagcaagggcttgctcagaaaagaccatttgatggtgaagacggtgctctggggcagcagcagtacctcactcggcttcgagagcttcaaagtgcatcagagaccagcctcgtcaatttcccaaaatccataccagtatcaggtacttccattcaaccaacccttggtgccaatggtgtgatattagacaaccagcctatagtcaaaaaaaggcgaggaaggaggaagaatgtagaaggtgttgacatcttcttttttaacagaaataaaccacctaatcatgtttctttaggcttaacctcctcacagatttccacagggataaatccagcactatcctatactcaacctcaaggaattcctgatacagaaagtccagttccagttattaatcttaaagatggaacgagacttgcaggagatgatgcaccaaagagaaaggatttggaaaaatggcttaaggagcacccgggttatgtggaagatttgggagcttttattcctagaatgcagcttcatgagggaagacccaaacaaaaaagacaccgttgcagaaaccccaataaactagatgtgaatagtctcactggagaagaacgtgttcaactgattaacagaagaaatgctagaaaggttggaggtgcatttgctccccctttgaaagatttatgtagattcctaaaagaaaattcagaatatggagtagctcctgaatggggagatgttgttaagcaatctggatttcttccagaaagcatgtatgaacgtattctcactggtcccgttgtgagagaggaagtaagcaggcgggggagacggcctaaaagtggaattgcaaaggccacagcagcagcagctgctgcatctgccaccagtgtttcaggcaatcctttgttagccaatggactacttccaggtgtggatctcacaactcttcaggccttacaacaaaacctacaaaacttgcagtcactgcaagtaactgctgggttgatgggaatgcctaccggccttccttctggaggagaagctaaaaacatggctgctatgttccccatgctgctgtcaggaatggctggattaccaaatctgttgggcatgggaggactcctgacaaagcctacggaatctgggacagaagacaaaaagggaagtgactctaaggagtcagaaggaaaaacagaaaggacagagagccaaagttcagagaatggtggagaaaactctgtgtcaagttctccttccgcatcctctactgctgcattaaatacagctgcagctgccaacccattagctcttaacccactattactatctaatatactttatccagggatgcttctcactccaggccttaatcttcatattccaactttgtcccagtccaatacttttgatgtacaaaacaaaaacagtgacttaggctcgtctaagtctgtagaagtaaaagaagaagattccagaattaaagatcaggaagacaaaggaggaactgaaccaagtcctctcaatgaaaacagcacagatgagggttcagagaaagctgatgcttcatctggatctgatagtacatcgtcgtcatctgaggattcagattctagtaatgaagactgattcccagactctgcacttaaaatatgaactgattttggattttttctttaataattaattgtaaataccccagtgttgagtgcatcaataacttactgaccgaacatttcagttatttgtttagaagtgcaaactgctttcagagactttttgcatgtaatatttcttaagattcataagtttctgaactcgtatgtactatcaaatacataaaggtgtaaaattacaacaaaaggcattataattttgttgggggttaattttatgaaaattatgctcaataagagttgtatatttaatatatttgcagtgaacacagaatactttatgcatattactgatttaatttgaatatagttttacagcctccttgacacctataatttacagatcaaaactcagcaataatttgggcagctaatgaatgtcatgaaagctgtagaatctacatcaccatccattgctttaattacatgaaaatgctctagtgttgtgatgcactgctgatgtttccaattcaggtacaagtatgttttaaagaagaaataagtttcccaatcagccaatttaactggctacctgttacctcagctgagttagtttaggaagtttacattcgtttctaattctatacttgttttcaggggttttttaaacacatcctatatatcatgtcaatctggcaagaaatatgacttgctttttgctgagcttaattcagatatcagtaaaattaagtcataaaataatcatgtgtcatgtgactttggcaccctatagacatacttagttttaacttttcaaagtttggcctcctattagaaataatcatgtctcagatgagtaatgtctgtttccagggttcagaaaaggcaaactcatgaaatgccactgaaaagaactttcaacacagcatacttcatgtaaaagaaattgtttgtttgctttctttgtgtagatttctatttgtgttttatgtcatggaaatattccagaattaacagataatagtggtaaagtaatatgcagatagtctaaattcattttgagtttctaggtgtaagcagactaaatgttgcccagaatcagtgttgggttatcagtttatattaaatatactgagttgcccgttttgaaaatgcactttgaataatctcaaaaagatgtacaagttatacctgtaaaccacaaaagtgaagcctgaggcttctgttcaatttcatagactcctttaccatgtaaaatttgtctgatatttgatttgtgatacaatttctcctgctaaagctgctattattctgacaaggtagaggtccaggttcacctttatatatatttaaaacaattagtactgaattggacataaaaatattgacattctaaggagagatatatgttagcatttttctggtactcaaataagttagtagtaaagtctgcaagggcataaatttagggggaaaaagtgtcccagttctctcctacagaaaaaatactttcagtatgttttgataaaactgttgctttgtcatgagttagtcaattgtatcaggttttccaagacctttaccagtaaattatgtttctgtatgtaaaataaccccttattagagagacagtgttatatgtatttacaaaattatataagttccattgggattgtattgattttgtattttcccaaaatagtactttgaattgatagtcctttatgcaatgtcttagcaatagtctctataatgcccatccaggagaagtgggtagtaattcttcatcatgaaaatgatatattacatatttagtatcttccctttgcagtattgcacttttgtttaactagaatacacctatgagatagccaaagtttcaaacacagttatcttagtttaccggtggagtatttcaacaccaaccacatttcccttcctccctctaattctacccacatgatctttattccttcctttcgccaattaaaaaaaaaaaaaaggaaaaaaaatctgtagatcttgtcactaaaatctaatttatatcaaatttatgagagaaagtattttcctaattatggtcaaataaatttggttaacatcctagtgattctctttctatataataaggcaattacagttttcaaagcattaagtctaacataactttaaacattctcttaggtttcaagacacttctatttaatattcattggggaaaagttgtccagctatcagctaagaaaacacatgcaaatatggttgtgtaaagttaagggttataaggaaaaaaaaatcagtagaattacataatactaaagttgcagttgaaagaatatccaagtatgtgttggtagttactaaaagaattatagctgttattgccttgtatttatagcccttgtttcaggttttatgattcaagtcttagtccaatctttcttttggacatttgcaatatttaccagttgtgttttgtgtagtctgaatttgctttctgtagttgagcaaacgtcttaaaaagtcatttgtaatttattaaattactttctatgatgttctatagagc

SEQ ID NO: 2 - protein, 2897 aa, Homo sapiens [sequence omitted]

SEQ ID NO: 3 - DNA, 4009 bp, Homo sapiens
  CDS (148)..(3558)
gaccaaaataaaagctttgtggcacaccatgactttgccttatttcaggccaatgaacaacaaacacagtgtacttcactacgctcacaacaaaacagaaataatctcaacccagggcagaattctcttagccagtctaaaaattttatgaatgtttctggtccacatagagtcaatgttaaccacccaccacaagatgaaaagcccaaactccggagaccctgtgaccgttccaatggctatggaagaactgaatgctttagagttgagaaaaacctgctagtttatgggtggggccgatggagagagattctatctcatggccgtttcaaaaggcagctaaatgaacacgatgtagagataatttgccgagctctcttagcatattgccttgttcactaccgaggagatgagaagattaaaggtttcatatgggatctcattactccaactgaagatggacagacacgagagctacagaatcatctaggcctatcagctcctgtacccaggggtcgaaaagggaagaaagtaaaaactcaaacaagctcatttgatatacaaaaagcagaatggcttcgaaaatataatcccgagcagctccttcaagatgaaggctacaaaaaacatataaaacaccactgtaataaggttttgcttcgtgtgagaatgctgtattatctaaagcaagaagttattggaaatgagtgtcagaaagtatttgatggagttgatgcaagtgacattgatgtttgggtaccagaaccagaccactcagaagttcctgctgagtggtgggattttgatgctgataagtcactccttattggagtttttaaacatggatatgaaaaatataacactattcgagcagacccagcattatgcttcttggaaagagtgggaaaacctgatgagaaagcagttgctgctgaacagagagcgaatgattatatggatggggatgtggaagatccagaatacaaacctgccccagccatctttaaagatgatatagaggatgatgtttcctcaccaggagatcttgttatagcagatggagatggtcaactgatggagggtgataaagtatattggcctactcaatcagctttaaccacacgtttgaggcgtctcatcactgcataccagcgtactaataaaaacagacaaattcaacagatacaaccgactttctcggtgcctaccagtgtaatgcagcctatttatgaggaagccactcttaatcctaaaatggcagccaagatagaaagacagcaaagatggacaagaagagaagaagctgacttttatagggttgtatctacatttggagtggtttttgaccctgacagaggccaatttgattggacaaaatttagagctatggctaggctacataagaaaactgatgatagtttggaaaaatatttgtacgcattcatgtccatgtgtcggagggtttgtcgtcttccttccaaagaagaaagtactactcacatgaaagcctatgatgaagaaagcgtcgcgtcactgagcactacccaggatgagactcaggatagttttcagatgaacaatgggacaccagagtctgcttatatcttacaaggtggatatatgctggcagcctcgtattggccaaaggatcgtgtgatgatcaataggttggacagtatttgtcaaacagttctgaaaggaaagtggccttcagctagaagaagttatgatgctaacacagtggcttctttctataccacaaaactgctggacagccctggagcagctacagaatacagcgagcccagtgtacccactcccccaggtgccggtgttaaagaagaacatgatcagtcaacacagatgtcaaaggtgaagaagcatgtacgagaaaaggagtttacagtgaaaatcaaagacgaaggtggtttgaagttgacatttcagaagcaagggcttgctcagaaaagaccatttgatggtgaagacggtgctctggggcagcagcagtacctcactcggcttcgagagcttcaaagtgcatcagagaccagcctcgtcaatttcccaaaatccataccagtatcaggtacttccattcaaccaacccttggtgccaatggtgtgatattagacaaccagcctatagtcaaaaaaaggcgaggaaggaggaagaatgtagaaggtgttgacatcttcttttttaacagaaataaaccacctaatcatgtttctttaggcttaacctcctcacagatttccacagggataaatccagcactatcctatactcaacctcaaggaattcctgatacagaaagtccagttccagttattaatcttaaagatggaacgagacttgcaggagatgatgcaccaaagagaaaggatttggaaaaatggcttaaggagcacccgggttatgtggaagatttgggagcttttattcctagaatgcagcttcatgagggaagacccaaacaaaaaagacaccgttgcagaaaccccaataaactagatgtgaatagtctcactggagaagaacgtgttcaactgattaacagaagaaatgctagaaaggttggaggtgcatttgctccccctttgaaagatttatgtagattcctaaaagaaaattcagaatatggagtagctcctgaatggggagatgttgttaagcaatctggatttcttccagaaagcatgtatgaacgtattctcactggtcccgttgtgagagaggaagtaagcaggcgggggagacggcctaaaagtggaattgcaaaggccacagcagcagcagctgctgcatctgccaccagtgtttcaggcaatcctttgttagccaatggactacttccaggtgtggatctcacaactcttcaggccttacaacaaaacctacaaaacttgcagtcactgcaagtaactgctgggttgatgggaatgcctaccggccttccttctggaggagaagctaaaaacatggctgctatgttccccatgctgctgtcaggaatggctggattaccaaatctgttgggcatgggaggactcctgacaaagcctacggaatctgggacagaagacaaaaagggaagtgactctaaggagtcagaaggaaaaacagaaaggacagagagccaaagttcagagaatggtggagaaaactctgtgtcaagttctccttccgcatcctctactgctgcattaaatacagctgcagctgccaacccattagctcttaacccactattactatctaatatactttatccagggatgcttctcactccaggccttaatcttcatattccaactttgtcccagtccaatacttttgatgtacaaaacaaaaacagtgacttaggctcgtctaagtctgtagaagtaaaagaagaagattccagaattaaagatcaggaagacaaaggaggaactgaaccaagtcctctcaatgaaaacagcacagatgagggttcagagaaagctgatgcttcatctggatctgatagtacatcgtcgtcatctgaggattcagattctagtaatgaagactgattcccagactctgcacttaaaatatgaactgattttggattttttctttaataattaattgtaaataccccagtgttgagtgcatcaataacttactgaccgaacatttcagttatttgtttagaagtgcaaactgctttcagagactttttgcatgtaatatttcttaagattcataagtttctgaactcgtatgtactatcaaatacataaaggtgtaaaattacaacaaaaggcattataattttgttgggggttaattttatgaaaattatgctcaataagagttgtatatttaatatatttgcagtgaacacagaatactttatgcatattactgatttaatttgaatatagttttacagcctccttgacacctataatttacagatcaaaactcagcaataatttgggcagctaatgaatgtcatgaaagctgtagaatcta

SEQ ID NO: 4 - protein, 1137 aa, Homo sapiens
MNVSGPHRVNVNHPPQDEKPKLRRPCDRSNGYGRTECFRVEKNLLVYGWGRWREILSHGRFKRQLNEHDVEIICRALLAYCLVHYRGDEKIKGFIWDLITPTEDGQTRELQNHLGLSAPVPRGRKGKKVKTQTSSFDIQKAEWLRKYNPEQLLQDEGYKKHIKHHCNKVLLRVRMLYYLKQEVIGNECQKVFDGVDASDIDVWVPEPDHSEVPAEWWDFDADKSLLIGVFKHGYEKYNTIRADPALCFLERVGKPDEKAVAAEQRANDYMDGDVEDPEYKPAPAIFKDDIEDDVSSPGDLVIADGDGQLMEGDKVYWPTQSALTTRLRRLITAYQRTNKNRQIQQIQPTFSVPTSVMQPIYEEATLNPKMAAKIERQQRWTRREEADFYRVVSTFGVVFDPDRGQFDWTKFRAMARLHKKTDDSLEKYLYAFMSMCRRVCRLPSKEESTTHMKAYDEESVASLSTTQDETQDSFQMNNGTPESAYILQGGYMLAASYWPKDRVMINRLDSICQTVLKGKWPSARRSYDANTVASFYTTKLLDSPGAATEYSEPSVPTPPGAGVKEEHDQSTQMSKVKKHVREKEFTVKIKDEGGLKLTFQKQGLAQKRPFDGEDGALGQQQYLTRLRELQSASETSLVNFPKSIPVSGTSIQPTLGANGVILDNQPIVKKRRGRRKNVEGVDIFFFNRNKPPNHVSLGLTSSQISTGINPALSYTQPQGIPDTESPVPVINLKDGTRLAGDDAPKRKDLEKWLKEHPGYVEDLGAFIPRMQLHEGRPKQKRHRCRNPNKLDVNSLTGEERVQLINRRNARKVGGAFAPPLKDLCRFLKENSEYGVAPEWGDVVKQSGFLPESMYERILTGPVVREEVSRRGRRPKSGIAKATAAAAAASATSVSGNPLLANGLLPGVDLTTLQALQQNLQNLQSLQVTAGLMGMPTGLPSGGEAKNMAAMFPMLLSGMAGLPNLLGMGGLLTKPTESGTEDKKGSDSKESEGKTERTESQSSENGGENSVSSSPSASSTAALNTAAAANPLALNPLLLSNILYPGMLLTPGLNLHIPTLSQSNTFDVQNKNSDLGSSKSVEVKEEDSRIKDQEDKGGTEPSPLNENSTDEGSEKADASSGSDSTSSSSEDSDSSNED

SEQ ID NO: 5 - DNA, 3854 bp, Homo sapiens
  CDS (402)..(1298)
ataaccttaacctttaatattccttctttattgttcttcccaattagaagggagccacttctttggtcttctaggacaagtgatggtgtgaaacatgtaagggttatggaagtacccatgaaaaaatttttttggaccttatattttctagaattatactacaatcttatgttttacagtatggaggaaggctaaagacctctttactcacttattctgtgataaaatgaaaagtggaagagctgtttgaagctaccacttatataaaattaggttattgctattataattgattatttaatatcagtacagaataaatttacaaataattacctatttttcatggttatttgtatgataagctaataactctttattttattttcatgtagggattattaatgaatatatatttcttccagggatttcttccagaaagcatgtatgaacgtattctcactggtcccgttgtgagagaggaagtaagcaggcgggggagacggcctaaaagtggaattgcaaaggccacagcagcagcagctgctgcatctgccaccagtgtttcaggcaatcctttgttagccaatggactacttccaggtgtggatctcacaactcttcaggccttacaacaaaacctacaaaacttgcagtcactgcaagtaactgctgggttgatgggaatgcctaccggccttccttctggaggagaagctaaaaacatggctgctatgttccccatgctgctgtcaggaatggctggattaccaaatctgttgggcatgggaggactcctgacaaagcctacggaatctgggacagaagacaaaaagggaagtgactctaaggagtcagaaggaaaaacagaaaggacagagagccaaagttcagagaatggtggagaaaactctgtgtcaagttctccttccgcatcctctactgctgcattaaatacagctgcagctgccaacccattagctcttaacccactattactatctaatatactttatccagggatgcttctcactccaggccttaatcttcatattccaactttgtcccagtccaatacttttgatgtacaaaacaaaaacagtgacttaggctcgtctaagtctgtagaagtaaaagaagaagattccagaattaaagatcaggaagacaaaggaggaactgaaccaagtcctctcaatgaaaacagcacagatgagggttcagagaaagctgatgcttcatctggatctgatagtacatcgtcgtcatctgaggattcagattctagtaatgaagactgattcccagactctgcacttaaaatatgaactgattttggattttttctttaataattaattgtaaataccccagtgttgagtgcatcaataacttactgaccgaacatttcagttatttgtttagaagtgcaaactgctttcagagactttttgcatgtaatatttcttaagattcataagtttctgaactcgtatgtactatcaaatacataaaggtgtaaaattacaacaaaaggcattataattttgttgggggttaattttatgaaaattatgctcaataagagttgtatatttaatatatttgcagtgaacacagaatactttatgcatattactgatttaatttgaatatagttttacagcctccttgacacctataatttacagatcaaaactcagcaataatttgggcagctaatgaatgtcatgaaagctgtagaatctacatcaccatccattgctttaattacatgaaaatgctctagtgttgtgatgcactgctgatgtttccaattcaggtacaagtatgttttaaagaagaaataagtttcccaatcagccaatttaactggctacctgttacctcagctgagttagtttaggaagtttacattcgtttctaattctatacttgttttcaggggttttttaaacacatcctatatatcatgtcaatctggcaagaaatatgacttgctttttgctgagcttaattcagatatcagtaaaattaagtcataaaataatcatgtgtcatgtgactttggcaccctatagacatacttagttttaacttttcaaagtttggcctcctattagaaataatcatgtctcagatgagtaatgtctgtttccagggttcagaaaaggcaaactcatgaaatgccactgaaaagaactttcaacacagcatacttcatgtaaaagaaattgtttgtttgctttctttgtgtagatttctatttgtgttttatgtcatggaaatattccagaattaacagataatagtggtaaagtaatatgcagatagtctaaattcattttgagtttctaggtgtaagcagactaaatgttgcccagaatcagtgttgggttatcagtttatattaaatatactgagttgcccgttttgaaaatgcactttgaataatctcaaaaagatgtacaagttatacctgtaaaccacaaaagtgaagcctgaggcttctgttcaatttcatagactcctttaccatgtaaaatttgtctgatatttgatttgtgatacaatttctcctgctaaagctgctattattctgacaaggtagaggtccaggttcacctttatatatatttaaaacaattagtactgaattggacataaaaatattgacattctaaggagagatatatgttagcatttttctggtactcaaataagttagtagtaaagtctgcaagggcataaatttagggggaaaaagtgtcccagttctctcctacagaaaaaatactttcagtatgttttgataaaactgttgctttgtcatgagttagtcaattgtatcaggttttccaagacctttaccagtaaattatgtttctgtatgtaaaataaccccttattagagagacagtgttatatgtatttacaaaattatataagttccattgggattgtattgattttgtattttcccaaaatagtactttgaattgatagtcctttatgcaatgtcttagcaatagtctctataatgcccatccaggagaagtgggtagtaattcttcatcatgaaaatgatatattacatatttagtatcttccctttgcagtattgcacttttgtttaactagaatacacctatgagatagccaaagtttcaaacacagttatcttagtttaccggtggagtatttcaacaccaaccacatttcccttcctccctctaattctacccacatgatctttattccttcctttcgccaattaaaaaaaaaaaaaaggaaaaaaaatctgtagatcttgtcactaaaatctaatttatatcaaatttatgagagaaagtattttcctaattatggtcaaataaatttggttaacatcctagtgattctctttctatataataaggcaattacagttttcaaagcattaagtctaacataactttaaacattctcttaggtttcaagacacttctatttaatattcattggggaaaagttgtccagctatcagctaagaaaacacatgcaaatatggttgtgtaaagttaagggttataaggaaaaaaaaatcagtagaattacataatactaaagttgcagttgaaagaatatccaagtatgtgttggtagttactaaaagaattatagctgttattgccttgtatttatagcccttgtttcaggttttatgattcaagtcttagtccaatctttcttttggacatttgcaatatttaccagttgtgttttgtgtagtctgaatttgctttctgtagttgagcaaacgtcttaaaaagtcatttgtaatttattaaattactttctatgatgttctatagagc

SEQ ID NO: 6 - protein, 298 aa, Homo sapiens [sequence omitted]

SEQ ID NO: 7 - DNA, 1862 bp, Homo sapiens
  CDS (196)..(1206)
gatatatggaagatttaaacaccattcattctttgtttttccacaaaggaccgtcctccttctaatatttggaagaaaatagatcaatccagggactataaaaatggcaatcaactcagggaatatcaactggaagggctcaactggctcttgttcaattggtacaatagacgaaactgcatcttagcagatgaaatgggtcttggcaaaactattcaatcaattacattcctctatgaaatccttctgactggtataagaggacctttcctgattattgctccactttctactattgcaaactgggagagagaatttcgtacgtggactgatattaacgttgtggtttatcatgggagcctgattagcagacaaatgatacagcaatacgagatgtacttcagggattcacaggggcgtatcattcgaggagcttacagattccaagccatcatcaccacttttgaaatgattcttggaggctgtggagagcttaatgcaattgaatggcgatgtgtgattattgatgaagcacataggttaaaaaataaaaattgtaaactcttagagggcctgaaactcatgaatctggaacacaaggtgcttttgactggcacccctctccaaaatacagttgaagaactatttagtcttcttcactttcttgaacccttaaggtttccttctgaatcaacatttatgcaagaatttggggatctggaaacagaggaacaggtacagaaacttcaggctatcctgaaaccaatgatgttgagacgattaaaagaagatgtggaaaagaagttggcacctaaagaagaaaccatcattgaagtagaacttactaatattcaaaagaaatactaccgggctatcttggaaaagaacttttcttttttatccaaaggagcaggacaaactaatgtacctaacttggtcaataccatgatggagctcaggaaatgttgtaatcatccatatcttataaaaggtgctgaggagaaaatacttggagaatttagagatacttacaatccagctgcttctgattttcatcttcaagcaatgatccagtctgctggtaaattggtccttattgataaattgcttcccaaaatgaaagccggaggtcataaagtgctcatcttctctcaaatggttcgttgccttgacattctggaggactatctcatacataaaaggtaaagcatactgaatttactgtgaattatgctaagtatatgctattaagatttgtgcagcactgtaatgtaaatgaactttactcatatctttctctttgctagattagaataatgcattcaaatttataagtaaagattgtttaggattatgtgttatattttcctaacctgcatatgttgcaatattagatgtgcctgaggagtccaattaagaaaagcttgcaaataaatatttgtttacccaggaatgggtttaaaaatgtacattttacttgattaataaaccttgtggatgaaactttaaatagatatatatttacaacagtcatcttcaaaattgaaaaaagtgattcaagaactttggatctatttcattgttataattagagccttaagttgtgggacttttattcttattatatactatataaaatttgttcatgctttaattttttctgatttgtttcaatgtgggttttttgttttgttttgtttattggtacatcacaataagatagtgttaaatattgtctaatcaggccgggcgcggtggctcacgtctgtaatcccagcactttgggagcctgaggcaggcggatcatgaggtcaggagatcaagaccatcgtggcttacacggtgaaacccacgtctctact

SEQ ID NO: 8 - protein, 336 aa, Homo sapiens
MGLGKTIQSITFLYEILLTGIRGPFLIIAPLSTIANWEREFRTWTDINVVVYHGSLISRQMIQQYEMYFRDSQGRIIRGAYRFQAIITTFEMILGGCGELNAIEWRCVIIDEAHRLKNKNCKLLEGLKLMNLEHKVLLTGTPLQNTVEELFSLLHFLEPLRFPSESTFMQEFGDLETEEQVQKLQAILKPMMLRRLKEDVEKKLAPKEETIIEVELTNIQKKYYRAILEKNFSFLSKGAGQTNVPNLVNTMMELRKCCNHPYLIKGAEEKILGEFRDTYNPAASDFHLQAMIQSAGKLVLIDKLLPKMKAGGHKVLIFSQMVRCLDILEDYLIHKR

SEQ ID NO: 9 - DNA, 20 bp, Artificial Sequence
  Synthetic
accacagtccatgccatcac

SEQ ID NO: 10 - DNA, 20 bp, Artificial Sequence
  Synthetic
tccaccaccctgttgctgta

SEQ ID NO: 11 - DNA, 15 bp, Artificial Sequence
  Synthetic
agcaacacagatgtc

SEQ ID NO: 12 - DNA, 21 bp, Artificial Sequence
  Synthetic
atcaggaattccttgaggttg

SEQ ID NO: 13 - DNA, 38 bp, Artificial Sequence
  Synthetic
gcgcaattgcgctcgacgattttttagcgcaattgcgc

SEQ ID NO: 14 - DNA, 32 bp, Artificial Sequence
  Synthetic
gatccatatatatatatatatatatatatgca

SEQ ID NO: 15 - protein, 9 aa, Homo sapiens
KKRRGRRKN

SEQ ID NO: 16 - protein, 9 aa, Homo sapiens
KKRRGRPPA

SEQ ID NO: 17 - protein, 9 aa, Homo sapiens
QKKRGRPPA

SEQ ID NO: 18 - protein, 9 aa, Homo sapiens
PKKRGRPRT

SEQ ID NO: 19 - protein, 9 aa, Homo sapiens
RKPRGRPKK

SEQ ID NO: 20 - DNA, 20 bp, Artificial Sequence
  Synthetic
gctaaaggagcacaatgcct

SEQ ID NO: 21 - DNA, 20 bp, Artificial Sequence
  Synthetic
ccccaaaaggaggacaaaat

SEQ ID NO: 22 - DNA, 20 bp, Artificial Sequence
  Synthetic
tagtacctccgcacgtggtc

SEQ ID NO: 23 - DNA, 20 bp, Artificial Sequence
  Synthetic
ctgcaggctcgagatagctt

SEQ ID NO: 24 - DNA, 21 bp, Artificial Sequence
  Synthetic
accagcctacaggctcttttt

SEQ ID NO: 25 - DNA, 20 bp, Artificial Sequence
  Synthetic
agagccagaccctgtctcaa

SEQ ID NO: 26 - DNA, 19 bp, Artificial Sequence
  Synthetic
aggctgcctttggtgactc

SEQ ID NO: 27 - DNA, 20 bp, Artificial Sequence
  Synthetic
ttataccctctgggctgtgc

SEQ ID NO: 28 - DNA, 25 bp, Artificial Sequence
  Synthetic
cgcatgatatacttcacctattttt

SEQ ID NO: 29 - DNA, 20 bp, Artificial Sequence
  Synthetic
ttgggctaggatatgcagaa

SEQ ID NO: 30 - DNA, 20 bp, Artificial Sequence
  Synthetic
aacagcctcctgtctaccga

SEQ ID NO: 31 - DNA, 20 bp, Artificial Sequence
  Synthetic
caggagaaaggagcatggac

SEQ ID NO: 32 - DNA, 204 bp, Homo sapiens
cgcatgatatacttcacctattttttgtctccttaatatgtatatatatatatatatatatatatatatatatatatatatatatatatatacacatatatgtgtgtgtgtatgtgcgtgtgcatgtttaacttttaattcagttaaaaacttttttctatttgtttttcatctggatatttgattctgcatatcctagcccaa

SEQ ID NO: 33 - DNA, 145 bp, Homo sapiens
gctaaaggagcacaatgcctgatgctgcaggagaaacattaggtaactatttaatggagttttaattttctgttattatttttaataattaattgtgattttgactatttggaagctacaggtatattttgtcctccttttgggg

SEQ ID NO: 34 - DNA, 113 bp, Homo sapiens
accagcctacaggctctttttaaatatttcccactttaggccggtacagtggcctaaaattttaatttttttcttccttttttttttttttttttgagacagggtctggctct

(19) United States
(12) Patent Application Publication
(10) Pub. No.: US 2005/0250717 A1
(43) Pub. Date: Nov. 10, 2005

(54) Chromatin remodeling protein as a marker expressed by stromal progenitor cells

(75) Inventors: Dafna Benayahu, Herzlia (IL); Irena Shur, Tel Aviv (IL)

Correspondence Address: BROWDY AND NEIMARK, P.L.L.C.;624 NINTH STREET, NW, SUITE 300, WASHINGTON, DC, 20001-5303, US

(73) Assignee: Ramot AT Tel Aviv University Ltd., Tel Aviv (IL)

(21) Appl. No.: 10/499,715

(22) PCT Filed: Dec. 20, 2002
(86) PCT No.: PCT/US02/40715

§ 371 Date: Oct. 28, 2004

Related U.S. Application Data
(60) Provisional application No. 60/341,831, filed on Dec. 21, 2001.

(51) Int. Cl. A61K 48/00; C12Q 1/68; C07H 21/04; C12N 9/22; C12N 15/09

(52) U.S. Cl. 514/44; 435/6; 435/69.1; 435/199; 435/320.1; 435/325; 536/23.2

(57) ABSTRACT

The present invention provides a chromatin remodeling protein, designated ChroM as well as alternatively spliced variants, which serves as markers for stromal precursor cells and osteogenic and muscle progenitor cells. This ChroM marker was found to be present in proliferating progenitor cells but is either absent or not predominantly present in resting cells. Antibodies directed against this ChroM marker can be used to isolate stromal progenitor cells that can differentiate into osteogenic and muscle cells. Subpopulations of human osteogenic progenitor cells can further be separated using additional markers. Due to the discovery that osteogenic progenitor sarcoma cells can be distinguished from other cells by the presence of ChroM in the nuclei or based on genetic alteration on the DNA level, a method for identifying osteosarcoma cells in a tissue sample and a method for evaluating the effectiveness of a treatment for osteogenic progenitor sarcoma are also provided.

### BACKGROUND OF THE INVENTION

1. Field of the Invention

The present invention relates to proteins involved with genome integrity and chromatin-remodeling complexes and their encoding DNA. The present invention also relates to antibodies against a chromatin remodeling protein which can be used to separate stromal mesenchymal including osteogenic and muscle progenitor cells from a cell mixture, to an isolated population of enriched cells, and to methods of using the chromatin remodeling protein and the isolated population of enriched cells.

2. Description of the Related Art

Transcriptional regulation of cell differentiation is a complex system and the multiple factors that play a role in the process of skeletal (bone and muscle) formation from mesenchymal precursors are not well sorted. Steroid hormones such as estrogen, androgen, glucocorticoids and vitamin D are known to play a major role in regulating physiological processes of the skeleton (Manolagas et al., 1999 and Spelsberg et al., 1999). Most important is their role relating to the ability of the hormone-bound nuclear receptors (NR) to change the expression of target genes in a cell- and promoter-dependent manner.

The transcriptional activity of NR depends on co-activators and co-repressors which regulate transcription by remodeling chromatin or by facilitating the recruitment of the basal transcriptional machinery. Co-activators are often part of multiprotein complexes which are not specific and which mediate the activity of other nuclear receptors (NRs). Surprisingly, different tissues respond in a selective manner to these hormones. Recent studies revealed that the activity of co-activators may contribute to the receptor, promoter and cell specificity of NR action (Jenkins et al., 2001 and Bourachot et al., 1999). The coordination of complex and dynamic networks in transcription regulation by steroid hormones is linked to other signaling pathways. A better understanding of the molecular mechanism underlying ligand-dependent transcriptional activation by nuclear receptors is believed to be through accessories proteins, co-activators and co-repressors. The co-activator proteins in complex with nuclear receptors act to remodel chromatin within the promoter region and to recruit the transcriptional machinery to the promoter in order to initiate transcription (Dilworth et al., 2000). The regulation of gene expression is based on transcription from chromatin templates. Chromatin remodeling occurs by the action of enzymes, which require ATP-dependent chromatin remodeling in proteins that are SNF-like, and in HAT enzyme or protein that possesses histone-binding domains, like the bromodomain, and contributes to the regulation of transcription by influencing activator-dependent recruitment (Fry et al., 2001). The specific activities of these proteins are still being explored and have become the focus of many research laboratories.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention is directed to a chromatin remodeling protein designated ChroMi, and fragments and variants thereof having the activity and properties of the chromatin remodeling protein, such as ATPase activity, DNA binding, LXXLL motif (NR box) for interaction with nuclear receptors, and GXXXG motif important in interactions with transmembrane (TM) proteins. The variants may be those having at least 95% sequence identity to the amino acid sequence of SEQ ID NO:2 or which are naturally occurring alternative splice forms.

The present invention also provides a nucleic acid molecule that contains the nucleotide sequence encoding ChroM1, or a fragment or variant thereof, and an antisense oligonucleotide that inhibits the production of the chromatin remodeling protein. The antisense oligonucleotide can be used in a method for treating osteosarcoma. Further provided is an isolated fragment of human genomic DNA which encodes ChroM1.

Another aspect of the present invention is a molecule having the antigen binding portion of an antibody specific for the chromatin remodeling protein of the present invention. This molecule can be used in a method for separating a cell population containing human stromal progenitor cells that differentiate into osteogenic and muscle cells from a cell mixture derived from human bone marrow by selectively binding the molecule to ChroM on human stromal progenitor cells. The antibody-bound cells are then separated from the cell mixture in to selective ex vivo culture conditions.

The method can be extended to further separate a subpopulation of human osteogenic progenitor cells through the use of additional markers present on the cells of the subpopulation. The present invention thus also provides an isolated cell population enriched for human osteogenic progenitor cells and a composition comprising a physiologically acceptable medium and the isolated cell population enriched for human osteogenic progenitor cells. A further aspect of the present invention relates to a method of treating bone or muscle tissue damage and a method of generating bone or muscle tissue.

Still further aspects of the present invention are directed to a method of screening for and identifying an enhancer or inhibitor compound that affect expression of the ChroM1 chromatin remodeling protein, to a method of identifying AT-rich promoter regions of genes involved in modulation of osteoblast differentiation and capable of binding to the DNA binding domain of ChroM1, and to a method of screening for and identifying a compound which stimulates differentiation-of osteogenic progenitor cells.

Due to the discovery that osteogenic progenitor sarcoma cells can be distinguished from normal osteoblasts by the presence of ChroM1 in the nuclei, the present invention further provides a method for identifying osteosarcoma cells in a tissue sample and a method for evaluating the effectiveness of a treatment, such as chemotherapy to specifically block the ChroM1 binding to chromatin and therefore block DNA binding in complex formation, for osteogenic sarcoma through the use of the molecule according to the present invention which has an antigen binding portion of an anti-ChroM antibody.

### BRIEF DESCRIPTION OF THE DRAWINGS

FIGS. 1A and 1B show schematic illustrations of the genomic organization of exons of ChroM1 by size and number (FIG. 1A). The functional protein motifs of ChroM1 are presented in FIG. 1B.

FIGS. 2 show the prediction for phosphorylation on amino acid residues serine and tyrosine. Immunoprecipitation (Ip) was performed with anti-ChroM antibody and analyzed by Western blot with anti-phosphotyrosine and anti-phosphoserine in the presence (+) or absence (−) of phosphatase inhibitors (Ph-I).

FIG. 3 shows a graph of the level of expression for ChroM1^{VE+} mRNA compared between cells derived from cultured trabecular bone (TBC) and those derived from marrow stromal cultured (MSC). The results demonstrate the distribution of ChroM1^{VE+} cells from different donors. The MSC and TBC cells are shown below the graph under the MSC and TBC labels.

FIG. 4 shows a graph of a RT-PCR analysis of the differential expression of ChroM1 in osteogenic/non-osteogenic clonal MSC population.

FIG. 5 shows in vivo staining of osteoprogenitor cells in tissue section and a corresponding schematic representation (reproduced from Alberts et al., Molecular Biology of the Cell).

FIG. 6 shows a graph of FACS quantification of bone marrow (TBM) and stroma cells (MSC) for level of expression of ChroM1 and visualization by immunohistochemical staining for MSC.

FIG. 7A shows FACS quantification of MSC (forward and side scatter), estimating the cell size and cytoplasmic granulation. The results expressed the ChroM1^{VE+}/ChroM1^{VE-} from the MSC divided into two subpopulations of small (S) and large (L) cells, ChroM1^{VE+} cells are in S region (FIG. 7A). In FIG. 7B, FACS analysis of the cell cycle based on DNA staining with PI, dot plot demonstrates double staining with PI versus membranous staining with FITC labeled anti ChroM antibody. 76% ChroM1^{VE+} cells are in GI phases and are of the small cell size, and the ChroM1^{VE+} expression on the cell surface is assessed for cell size and cycle.

FIG. 8A shows a graph of a FACS analysis of double staining with cell surface ChroM1 and with intracellular cell cycle markers (c-Fos, cjun, Ki-67, cyclinD1 and B1). Data is presented as percent of positively stained cells for ChroM1 and each of the markers (mean±SD) obtained from at least three experiments. FIG. 8B is a schematic illustration of the presence of the intracellular cell cycle markers at different stages in the cell cycle.

FIGS. 9A and 9B show the results of a FACS analysis for ChroM1 expression in cultured cells from four donors. 1×10⁵ MSC were plated in 100 mm culture dish and grown for a week in the growth medium (10% FCS in DMEM). The plates were then replaced with fresh medium contained 2% FCS (low serum) or 10% FCS (high serum) for an additional week. Cells were then released with EDTA and subjected to analysis by FACS for ChroM1 expression.

FIG. 10 shows a graph of an analysis of cell surface markers. The results represent the co-expression of ChroM with one of the selected markers: CD44, CD51, CD61, CD62E, CD62L, CD62P and CD34. The double staining analysis results are shown by histograms representing the expression of ChroM1^{VE+} to a specific marker or negative cells. The analysis was performed for different donors n=6.

FIG. 11 shows a graph of ATPase activity in IP, for 3 different donors, calculated as an average±SD of triplicates in each experiment. A calibration curve of serial concentrations of KHPO₄ was used to determine the amount of Pi released in the colorimetric assay.

FIG. 12A shows multiple alignment of KR region (DNA binding domain of HMGI/Y; SEQ ID NO:19) in ChroM1 (SEQ ID NO:15), BRM (SEQ ID NO:16), BRG1 (SEQ ID NO:17) and CHD1 (SEQ ID NO:18) and FIG. 12B shows an EMSA gel that represents the mobility of ³²P-labeled oligonucleotide in the presence and absence of rP (2-1 μg, 3-3 μg 4-3μg and 100× excess of unlabeled oligonucleotide).

FIG. 13 shows the IP results from biotinylated cell membrane and ChIP extraction on gel stained with Coomassie and onWestern blot.

FIG. 14 The ChIP analysis of primary MSC cells from 3 human donors followed with PCR amplification for three promoters at two regions (P, proximal and D, distal). Lane 1 is control and lanes 2 and 3 are treated cells in the presence of 17-β estradiol or TGFβ, respectively.

FIGS. 15A and 15B show PCR amplification from ChIP DNA for EMSA analysis of estrogen receptor promoter (ER; FIG. 15A) and BMP4 promoter (FIG. 15B) where: − indicates probe only; + indicates with recombinant protein (rP); and D indicates in the presence of distamycin A.

FIGS. 16A-16G show immunohistochemical staining of mouse embryo sections at day 16 and day 4 post-natal. The staining is in mesenchymal condensation during skeletal development.

FIG. 17 shows the immunohistochemistry of mouse sections where positive staining is detected in progenitors at the skeletal muscle, which are suggested as satellite cells.

FIGS. 18A-18D show the histopathology of normal human bone section and tumors in comparison to cultured cells. The ChroM1 expression in tissue section of normal bone (FIG. 18A) or cultured MSC was membranous or cytoplasm (FIG. 18B). Biopsies of osteogenic sarcoma (FIG. 18C) expressed a different cellular pattern, where the protein was translocated to the nucleus (FIG. 18D).

FIG. 19 shows schematic illustrations of alternative splice forms for ChroM1, ChroM2, and ChroM3 and hypothetical protein FLJ12178 by showing exon size and number.

FIGS. 20A-20D show schematic illustrations of open reading frames (ORFs) in different frames for the splice forms, ChroM1 (FIG. 20A), ChroM3 (FIG. 20B), ChroM2 (FIG. 20C), and hypothetical protein FLJ12178 (FIG. 20D). The representations are the same as presented in FIGS. 1 and 19A-19D.

### DETAILED DESCRIPTION OF THE INVENTION

The present invention is based on the discovery by the present inventors of a novel chromatin remodeling protein, ChroM1, which is present in stromal precursor cells/osteogenic and muscle cells. Bioinformatic analysis showed that other known proteins that have similar domain motifs or combinations of domains are involved with steroid receptors and in chromatin remodeling. ChroM1 has ATPase activity and DNA Binding Domain (DBD) that was found to co-precipitate with DNA. Using anti-ChroM1 antibodies, the laboratory of the present inventors found ChroM1 by FACS and by immunohistochemistry in osteoblastic cells at the cell membrane and in the cytoplasm with negligible amounts in the nucleus. It is expected that ChroM1 is a co-factor that is part of a complex system of chromatin regulation in association with the steroid hormones (SR) such as estrogen and glucocorticoid receptors that allow the opening up of chromatin for access by the steroid and for initiating the start of the transcription process. Transcription factors have restricted access to DNA, and they are affected locally by chromatin remodeling factors, which have a key role in differential gene expression.

In addition, it appears that the ChroM1 protein is part of the SWI/SNF2 subfamily, which is known to be a marker of embryonic development. From the results obtained in mouse development, it was found that ChroM1 as a marker appears to be directly related to skeletal development. It is known that skeletal development begins at 16.5 days in the mouse, and the presence of the marker coincides exactly with the beginning of skeletal development.

The laboratory of the present inventors has also found that this ChroM1 marker is present in proliferating cells but is either absent or not predominantly present in resting cells. ChroM1 is expressed with other markers of early differentiation such as CD34, CD44, selectin receptors and integrin receptors.

ChroM1 has the amino acid sequence of SEQ ID NO:2. While ChroM1 is a preferred embodiment of the polypeptide according to the present invention, it is intended that the polypeptide comprehend a fragment of ChroM1, a variant of ChroM1 having at least 95% sequence identity, preferably at least 98% sequence identity, to the amino acid sequence of SEQ ID NO:2, and a fragment of this variant provided that such a polypeptide has the activity of a chromatin remodeling protein, i.e., ATPase activity and DNA binding domain, etc.

Naturally-occurring alternative splice variants of ChroM1 were also discovered by the inventor and are considered to be part of the present invention. Accordingly, the present invention includes three specific naturally occurring alterative splice variants ChroM2 (SEQ ID NO:4), ChroM3 (SEQ ID NO:6), and a hypothetical protein FLJ12178 (SEQ ID NO:8).

One aspect of the present invention also relates to a composition which contains one or a combination of the polypeptide of the present invention and the naturally-occurring alternative splice variants thereof. This composition further includes an excipient, diluent, carrier or auxiliary agent, which is preferably pharmaceutically acceptable.

The present invention is also directed to a molecule having the antigen-binding portion of an antibody which binds to a polypeptide according to the present invention. The polypeptide of the present invention is preferably ChroM1 and the binding is preferably with high specificity.

It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)₂ fragments. Furthermore, the DNA encoding the variable region of the antibody can be inserted into the DNA encoding other antibodies to produce chimeric antibodies (see, for example, U.S. Pat. No. 4,816,567). Single chain antibodies can also be produced and used. Single chain antibodies can be single chain composite polypeptides having antigen binding capabilities and comprising a pair of amino acid sequences homologous or analogous to the variable regions of an immunoglobulin light and heavy chain (linked V_{H}-V_{L} or single chain F_{V}). Both V_{H} and V_{L} may copy natural monoclonal antibody sequences or one or both of the chains may comprise a CDR-FR construct of the type described in U.S. Pat. No. 5,091,513, the entire contents of which are hereby incorporated herein by reference. The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are hereby incorporated herein by reference.

A “molecule having the antigen-binding portion of an antibody” as used herein is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, Fab miniantibodies (see WO 93/15210, U.S. patent application Ser. No. 08/256,790, WO 96/13583, U.S. patent application Ser. No. 08/817,788, WO 96/37621, U.S. patent application Ser. No. 08/999,554, the entire contents of which are incorporated herein by reference) and chimeric or single-chain antibodies incorporating such reactive fraction, as well as any other type of molecule in which such antibody reactive fraction has been physically inserted. Such molecules may be provided by any known technique, including, but not limited to, enzymatic cleavage, peptide synthesis or recombinant techniques.

The term “epitope” is meant to refer to that portion of any molecule capable of being bound by an antibody which can also be recognized by that antibody. Epitopes or antigenic determinants usually consist of chemically active surface groupings of molecules such as amino acids or sugar side chains and have specific three-dimensional structural characteristics as well as specific charge characteristics.

An “antigen” is a molecule or a portion of a molecule capable of being bound by an antibody which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

Monoclonal antibodies (mAbs) are a substantially homogeneous population of antibodies to specific antigens. MAbs may be obtained by methods known to those skilled in the art. See, for example Kohler et al (1975); U.S. Pat. No. 4,376,110; Ausubel et al (1987-1999); Harlow et al (1988); and Colligan et al (1993), the contents of which references are incorporated entirely herein by reference. Such antibodies may be of any immunoglobulin class including IgG, IgM, IgE, IgA, and any subclass thereof. The hybridbma producing the mAbs of this invention may be cultivated in vitro or in vivo. High titers of mAbs can be obtained in in vivo production where cells from the individual hybridomas are injected intraperitoneally into pristane-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

Chimeric antibodies are molecules, the different portions of which are derived from different animal species, such as those having a variable region derived from a murine mAb and a human immunoglobulin constant region. Antibodies which have variable region framework residues substantially from human antibody (termed an acceptor antibody) and complementarity determining regions substantially from a mouse antibody (termed a donor antibody) are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (Better et al, 1988; Cabilly et al, 1984; Harlow et al, 1988; Liu et al, 1987; Morrison et al, 1984; Boulianne et al, 1984; Neuberger et al, 1985; Sahagan et al (1986); Sun et al, 1987; Cabilly et al, European Patent Application 125023 (published Nov. 14, 1984); Taniguchi et al, European Patent Application 171496 (published Feb. 19, 1985); Morrison et al, European Patent Application 173494 (published Mar. 5, 1986); Neuberger et al, PCT Application WO 8601533, (published Mar. 13, 1986); Kudo et al, European Patent Application 184187 (published Jun. 11, 1986); Morrison et al., European Patent Application 173494 (published Mar. 5, 1986); and Robinson et al., International Patent Publication WO 9702671 (published May 7, 1987) Queen et al., (1989) and WO 90/07861, U.S. Pat. Nos. 5,693,762, 5,693,761, 5,585,089, 5,530,101 and Winter, U.S. Pat. No. 5,225,539, and WO 92/22653. These references are hereby incorporated by reference.

Besides the conventional method of raising antibodies in vivo, antibodies can be produced in vitro using phage display technology. Such a production of recombinant antibodies is much faster compared to conventional antibody production and they can be generated against an enormous number of antigens. By contrast, in the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies one can use various methods all based on phage display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

Due to the discovery that ChroM1 is present in human osteogenic progenitor cells and proliferating osteoblasts, but not in mature osteoblasts, ChroM1 can be used as a marker for distinguishing the partially differentiated osteogenic progenitor cells and proliferating osteoblasts from mature osteoblasts and other non-ChroM-containing cells. For instance, bone marrow, which is the soft tissue occupying the medullary cavities of long bones, some haversian canals, and spaces between trabeculae of cancellous or spongy bone, is a source of osteogenic progenitor cells and proliferating osteoblasts. However, in order to isolate an enriched population of osteogenic progenitor cells and proliferating osteoblasts from a cell mixture obtained from bone marrow, a procedure for selectively recovering the osteogenic progenitor cells and proliferating osteoblasts is needed.

Osteogenic progenitor cells and proliferating osteoblasts can be separated from other cells by virtue of the presence of ChroM1 expressed on these cells. A molecule containing the antigen binding portion of an antibody specific for ChroM1, which molecule is preferably a polyclonal anti-ChroM1 antibody, can be used to enrich for a cell population of ChroM1 positive osteogenic progenitor cells and proliferating osteoblasts from a cell mixture. Thus, the present invention provides a method for separating a cell population containing human osteogenic progenitor cells and proliferating osteoblasts from a cell mixture derived from a source of these cells, such as bone marrow. This method involves contacting the cell mixture with a molecule having the antigen binding portion of an antibody specific for ChroM1, which molecule selectively binds to a ChroM1 antigen on human osteogenic progenitor cells and proliferating osteoblasts. The antibody-bound cells are then separated from the cell mixture.

The cells can be isolated by conventional techniques for separating cells, such as those described in Civin, U.S. Pat. Nos. 4,714,680, 4,965,204, 5,035,994, and 5,130,144, Tsukamoto et al 5,750,397, and Loken et al, U.S. Pat. No. 5,137,809, all of which are hereby incorporated by reference in their entirety. Thus, for example, a ChroM1-specific polyclonal antibody can be immobilized, such as on a column or on magnetic beads. The entire cell mixture may then be passed through the column or added to the magnetic beads. Those which remain attached to the column or are attached to the magnetic beads, which may then be separated magnetically, are those cells which contain a marker which is recognized by the antibody used. Thus, if the anti-ChroM1 antibody is used, then the resulting population will be greatly enriched in ChroM1 positive cells.

Another way to sort progenitor cells is by means of flow cytometry, most preferably by means of a fluorescence-activated cell sorter (FACS), such as those manufactured by Becton-Dickinson under the names FACScan or FACSCalibur. By means of this technique, the cells having a ChroM1 marker thereon are tagged with a particular fluorescent dye by means of an anti-ChroM1 antibody (or more generally a molecule having the antigen binding of an anti-ChroM1 antibody) which has been conjugated to such a dye. Similarly, another marker for a specific subpopulation of osteogenic progenitor cells are tagged with a different fluorescent dye by means of an antibody against this second marker which is conjugated to the other dye. When the stained cells are placed on the instrument, a stream of cells is directed through an argon laser beam that excites the fluorochrome to emit light. This emitted light is detected by a photo-multiplier tube (PMT) specific for the emission wavelength of the fluorochome by virtue of a set of optical filters. The signal detected by the PMT is amplified in its own channel and displayed by a computer in a variety of different forms e.g., a histogram, dot display, or contour display. Thus, fluorescent cells which emit at one wavelength, express a molecule that is reactive with the specific fluorochrome-labeled reagent, whereas non-fluorescent cells or fluorescent cells which emit at a different wavelength do not express this molecule but may express the molecule which is reactive with the fluorochrome-labeled reagent which fluoresces at the other wavelength. The flow cytometer is also semi-quantitative in that it displays the amount of fluorescence (fluorescence intensity) expressed by the cell. This correlates, in a relative sense, to the number of the molecules expressed by the cell.

Flow cytometers are also equipped to measure non-fluorescent parameters, such as cell volume or light scattered by the cell as it passes through the laser beam. Cell volume is usually a direct measurement. The light scatter PMTs detect light scattered by the cell either in a forward angle (forward scatter; FSC) or at a right angle (side scatter; SSC). FSC is usually an index of size, whereas SSC is an index of cellular complexity, although both parameters can be influenced by other factors.

Preferably, the flow cytometer is equipped with more than one PMT emission detector. The additional PMTs may detect other emission wavelengths, allowing simultaneous detection of more than one fluorochrome, each in individual separate channels. Computers allow the analysis of each channel or the correlation of each parameter with another. Fluorochromes which are typically used with FACS machines include fluorescein isothiocyanate (FITC), which has an emission peak at 525 nm (green), R-phycoerythrin (PE), which has an emission peak at 575 nm (orange-red), propidium iodide (PI), which has an emission peak at 620 nm (red), 7-aminoactinomycin D (7-AAD), which has an emission peak at 660 nm (red), R-phycoerythrin Cy5 (RPE-Cy5), which has an emission peak at 670 nm (red), and allophycocyanin (APC), which has an emission peak at 655-750 nm (deep red).

These and other types of FACS machines may have the additional capability to physically separate the various fractions by deflecting the cells of different properties into different containers.

Any other method for isolating the osteogenic progenitor cells as an enriched population from a cell mixture as a starting material, such as bone marrow, may also be used in accordance with the present invention. The various subpopulations of the present invention may be isolated in a similar manner.

Certain additional markers can be used to differentiate between subpopulations of osteogenic progenitor cells and proliferating osteoblasts, such as CD44, CD51, CD61, CD62 (E, L, P), CD34, etc.

By contacting either enriched anti-ChroM antibody-bound cells or the starting cell mixture with a second antibody which selectively binds a second antigen in a subpopulation of human osteogenic progenitor cells and proliferating osteoblasts, the subpopulation of human osteogenic progenitor cells can be separated following a first enrichment for ChroM1 positive cells.

A further aspect of the present invention is directed to an isolated cell population enriched for human stromal progenitors. For example, proliferating osteoprogenitors, preferably obtained using the methods of separating progenitor cells and proliferating osteoblasts from a cell mixture according to the present invention. The isolated cell population may be additionally enriched for those progenitor cells that can differentiate into muscle cells. These enriched cell populations may be used for tissue engineering of skeletal muscle, cardiac and bone tissues. Preferably, the human osteogenic progenitor cells that can differentiate into osteogenic cells and muscle cells according to the present invention are not human embryonic stem cells.

While osteogenic progenitor cells and proliferating osteoblasts can be isolated in substantial purity, i.e., in a substantially homogeneous population greater than 50%, preferably greater than 60%, more preferably greater than 70% cells which are ChroM1 positive, by the methods discussed above, such as, for example, by means of the FACS apparatus, it is not always necessary that the ChroM1 positive osteogenic and proliferating osteoblast cell population of the present invention be present in substantial purity. For example, the present invention also comprehends an isolated population of cells containing greater than 40% cells which are positive for ChroM1. Normally, cell populations containing human osteogenic progenitor cells sampled from the body contain less than 10% cells positive for ChroM1. Such a low purity subpopulation still defines over the prior art and yet maintains many of the advantages of the present invention. Isolated cell populations having greater than 30% of ChroM1 positive cells are also considered to be part of the present invention. A further aspect of the present invention is directed to a composition which contains a physiologically acceptable medium and the isolated cell population of cells according to the present invention.

The isolated or separated human osteogenic progenitor cells and proliferating osteoblasts of the present invention, whether or not obtained using the method according to the present invention, can be expanded in number by long term in vitro culture with minimal differentiation.

The pluripotent progenitors can be induced to differentiate under various culture conditions.

The localization of ChroM1 in different cellular compartments such as the cell membrane, cytoplasm, and the nucleus provides a connection between the extracellular environment and transcription. Tissue specific progenitor cells can proliferate in vivo in the appropriate niche only. Thus in vitro expanded mesenchymal cells can be used for the recovery of defects in bone, skeletal and cardio-muscle. This may be affected by the immediate regulation and genomic effect of steroid hormones in a tissue and cell specific manner.

After population expansion in culture, isolated stromal progenitor cells can then be harvested and activated to differentiate under various conditions, such as mechanical, cellular, and biochemical stimuli. By activating stromal progenitors to differentiate into the specific types of cells desired, such as bone-forming osteoblast cells, etc., a highly effective process exists for treating skeletal and other connective tissue disorders.

The culture medium can also contain additional components, such as osteoinductive factors. The osteoinductive factors include any that are now known and any factors which are later recognized to have osteoinductive activity. Such osteoinductive factors include, for example, dexamethasone, ascorbic acid-2-phosphate, β-glycerophosphate and TGF superfamily proteins, such as the bone morphogenic proteins BMPS. The presence of bioactive factors such as dexamethasone, ascorbic acid-2-phosphate and β-glycerophosphate in the culture medium directs human MSCs into the osteogenic lineage. The presence of a bioactive factor such as 5-azacytidine, 5-azadeoxycytidine, or analogs of either of them in the culture medium directs human mesenchymal stem cells into the myogenic lineage.

As a result, a process has been developed for isolating and purifying human osteogenic progenitor cells from tissue prior to differentiation and then culture expanding the osteogenic progenitor cells to produce a valuable tool for musculoskeletal therapy. The objective of such manipulation is to greatly increase the number of osteogenic progenitor cells and to utilize these cells to redirect and/or reinforce the body's normal reparative capacity. The osteogenic progenitor cells are expanded to great numbers and applied to areas of connective tissue damage to enhance or stimulate in vivo growth for regeneration and/or repair, to improve implant adhesion to various prosthetic devices through subsequent activation and differentiation, etc.

Along these lines, various procedures are contemplated, as would be well appreciated by those of skill in the art, for transferring, immobilizing, and activating the culture-expanded, purified osteogenic progenitor cells at the site for repair, implantation, etc., including injecting the cells at the site of a skeletal defect, incubating the cells with a prosthesis and implanting the prosthesis, etc. Thus, by isolating, purifying and greatly expanding the number of cells prior to differentiation and then actively controlling the differentiation process by virtue of their positioning at the site of tissue damage or by pre-treating in vitro prior to their transplantation, the culture-expanded, osteogenic progenitor cells can be utilized for various therapeutic purposes such as to alleviate cellular, molecular, and genetic disorders in a wide number of metabolic bone diseases, skeletal dysplasias and other musculoskeletal and connective tissue disorders.

In the context of tissue engineering and skeletal and cardiac tissue repair, tissue regeneration therapy is the local application of autologous (host-derived) and allogeneic (non-host derived) cells to promote reconstruction of tissue defects caused by trauma, disease or surgical procedures. The objective of the tissue regeneration therapy approach is to deliver high densities of repair-competent cells (or cells that can become competent when influenced by the local environment) to the defect site in a format that optimizes both initial wound mechanics and eventual neo-tissue production. For soft tissue repair, it is likely that an implant vehicle(s), such as a matrix or scaffold, will be required to 1) transport and constrain the autologous cells in the defect site and 2) provide initial mechanical stability to the surgical site. In an optimal system, it is likely that the vehicle will slowly biodegrade at a rate comparable to the production of neo-tissue and development of strength in the reparative tissue (Goodship et al., 1986).

One aspect of the present invention provides a method of treating bone or muscle tissue damage involving administering to a patient in need thereof with bone or muscle tissue damage the isolated population of human cells enriched for human osteogenic progenitor cells according to the present invention. These cells are preferably culture expanded and are preferably autologous to the patient to which they are administered. In addition, the administered cells can be administered as part of an implant vehicle such as a matrix or scaffold for tissue regeneration.

A further aspect of the present invention provides a method of generating bone or muscle tissue, such as cardio-muscle, which involves seeding a matrix or scaffold, that can be used as an implant vehicle in a patient, with the isolated population of cells enriched for human osteogenic progenitor cells according to the present invention. The seeded cells are preferably culture expanded either before or after seeding the matrix or scaffolding. Non-limiting examples of matrices or scaffolding suitable for tissue engineering/regeneration are taught in U.S. Pat. Nos. 6,365,149; 6,200,606; 5,939,323; 6,323,146; 6,323,278; 5,893,888; and 6,228,117, the contents of which are herein incorporated by reference.

A still further aspect of the invention relates to the discovery that ChroM1 is translocated to the nucleus in osteogenic sarcoma cells. Therefore osteosarcoma cells can be identified based on staining of ChroM1 in the nuclei of osteosarcoma cells using anti-ChroM1 antibodies to indicate their degree of malignancy; the nuclei in osteosarcoma cells are darkly stained using anti-ChroM1 antibodies. By contrast, the nuclei of non-osteosarcoma cells are not stained. As will be appreciated by those in the art, this discovery can also be extended to using differential staining with anti-ChroM1 antibodies to the nuclei of osteosarcoma cells in a method for evaluating the effectiveness of a treatment for osteogenic sarcoma. The method involves contacting a tissue sample, i.e., biopsy, containing osteogenic cells with a molecule having an antigen-binding portion of an anti-ChroM1 antibody and detecting the presence of the molecule in the nuclei of cells in the tissue sample to identify osteosarcoma cells and to evaluate the effectiveness of the treatment.

A further aspect of the present invention is directed to a nucleic acid molecule containing a nucleotide sequence encoding any of the polypeptides according to the present invention, which preferably comprise the amino acid sequence of SEQ ID NO:2 (ChroM1), or the specific splice variant polypeptides ChroM2 (SEQ ID NO:4), ChroM3 (SEQ ID NO:6), and hypothetical protein FLJ 12178 (SEQ ID NO:8), a hypothetical alternative ChroM splice form. More specifically, the nucleic acid molecule contains a nucleotide sequence coding for ChroM which corresponds to nucleotides 88 to 8781 of SEQ ID NO:1, or a nucleotide sequence coding for any of the alternative splice variants corresponding to nucleotide 447 to 3728 of SEQ ID NO:3, nucleotide 402 to 1298 of SEQ ID NO:5, or nucleotide 196 to 1206 of SEQ ID NO:7.

Also comprehended by the present invention are nucleic acid molecules encoding alternative splice variants which are based on computer analysis and cloning by RT-PCR (Table 10), and nucleic acid molecules which hybridizes to the nucleotide sequence corresponding to 88 to 8781 of SEQ ID NO:1 under high stringency conditions.

Stringency conditions are a function of the temperature used in the hybridization experiment and washes, the molarity of the monovalent cations in the hybridization solution and in the wash solution(s) and the percentage of formamide in the hybridization solution. In general, sensitivity by hybridization with a probe is affected by the amount and specific activity of the probe, the amount of the target nucleic acid, the detectability of the label, the rate of hybridization, and the duration of the hybridization. The hybridization rate is maximized at a Ti (incubation temperature) of 20-25° C. below Tm for DNA:DNA hybrids and 10-15° C. below Tm for DNA:RNA hybrids. It is also maximized by an ionic strength of about 1.5M Na⁺. The rate is directly proportional to duplex length and inversely proportional to the degree of mismatching.

Specificity in hybridization, however, is a function of the difference in stability between the desired hybrid and “background” hybrids. Hybrid stability is a function of duplex length, base composition, ionic strength, mismatching, and destabilizing agents (if any).

The Tm of a perfect hybrid may be estimated for DNA:DNA hybrids using the equation of Meinkoth et al (1984), as
Tm=81.5° C.+16.6 (log M)+0.41 (% GC)−0.61 (% form)−500/L
and for DNA:RNA hybrids, as
Tm=79.8° C.+18.5 (log M)+0.58 (% GC)−11.8 (% GC)²−0.56(% form)−820/L where
M, molarity of monovalent cations, 0.01-0.4 M NaCl, % GC, percentage of G and C nucleotides in DNA, 30%-75%, % form, percentage formamide in hybridization solution,
and L, length hybrid in base pairs.

Tm is reduced by 0.5-1.5° C. (an average of 1° C. can be used for ease of calculation) for each 1% mismatching.

The Tm may also be determined experimentally. As increasing length of the hybrid (L) in the above equations increases the Tm and enhances stability, the full-length-rat gene sequence can be used as the probe.

Filter hybridization is typically carried out at 68° C., and at high ionic strength (e.g., 5-6×SSC), which is non-stringent, and followed by one or more washes of increasing stringency, the last one being of the ultimately desired high stringency. The equations for Tm can be used to estimate the appropriate Ti for the final wash, or the Tm of the perfect duplex can be determined experimentally and Ti then adjusted accordingly.

Hybridization conditions should be chosen so as to permit allelic variations, but avoid hybridizing to other genes. In general, stringent conditions are considered to be a Ti of 5° C. below the Tm of a perfect duplex, and a 1% divergence corresponds to a 0.5-1.5° C. reduction in Tm. Typically, rat clones were 95-100% identical to database rat sequences, and the observed sequence divergence may be artifactual (sequencing error) or real (allelic variation). Hence, use of a Ti of 5-15° C. below, more preferably 5-10° C. below, the Tm of the double stranded form of the probe is recommended for probing a rat cDNA library with a rat DNA probe or a human cDNA library with a human DNA probe.

As used herein, highly stringent conditions are those which are tolerant of up to about 5% sequence divergence. Without limitation, examples of highly stringent (5-10° C. below the calculated Tm of the hybrid) and moderately stringent (15-10° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the washing conditions, particularly if the hybridization conditions used are those which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE), 5× Denhardt's reagent, 0.5% SDS, 100 μg/ml denatured, fragmented salmon sperm DNA at an appropriate incubation temperature Ti.

The present invention also provides a vector containing the nucleic acid molecule according to the present invention and a host cell transformed with the nucleic acid molecule of the present invention. Moreover, the present invention provides a process for producing or preparing a polypeptide or a variant thereof according to the present invention. This process involves artificially/recombinantly expressing the polypeptide or a variant thereof in a recombinant host cell, where the nucleotide sequence encoding the polypeptide or a variant thereof is operably-linked to a promoter suitable for driving expression of the polypeptide or variant thereof in the host cell. The expressed polypeptide is then recovered to prepare the polypeptide or variant thereof.

The present invention further provides the identification of the genomic localization for ChroM. The ChroM gene was mapped to chromosome 16q12.2 using the radiation hybrid panel RH-G3 (Stanford Human Genome Center). The localization was confirmed with the advancement of the human genome project and localized to the contigs AC007345, AC007906. It would be well appreciated by those of skill in the art that the genomic ChroM gene can be isolated based on this genome localization.

Overexpression/overproduction of ChroM1 may contribute to an abnormality found in the disease osteopetrosis. Accordingly, an aspect of the present invention also relates to a method for treating osteopetrosis by inhibiting the production (overproduction) of ChroM1. A preferred embodiment of this method involves administering an antisense oligonucleotide, which inhibits production of ChroM1 and its activity as a chromatin remodeling protein, to a patient in need thereof. The antisense oligonucleotide is complementary to a messenger RNA (mRNA), encoding ChroM1 which includes nucleotides 88 to 8781 of SEQ ID NO:1.

The ChroM1 protein of the present invention is involved with genome integrity and the control of chromatin remodeling complexes that facilitate gene expression by helping transcription factors gain access to their DNA targets in chromatin. The present inventors believe that ChroM1 plays a role in the assembly and activity of the nuclear receptor (NR) transcription complex and serves as a co-factor which is part of a complex system of chromatin regulation in association with steroid hormone receptors (SR), such as estrogen and glucocorticoid receptors, that allows opening up of chromatin for access by the steroid and for initiating the start of the transcription process.

It is also believed that reduced expression of ChroM1 may contribute to a reduction in osteogenic progenitor cell activity, such as found in osteoporosis. Accordingly, another aspect of the present invention relates to a method for treating osteoporosis by inducing the expression of ChroM1. A preferred embodiment of this method involves pharmacological modulation to induce or enhance production of ChroM1 and its activity as a chromatin remodeling protein, such as by administering to a patient in need thereof a compound that enhances/induces the expression of ChroM1 as screened and identified by the method described below.

The present invention provides for a method of screening for and identifying an enhancer or inhibitor compound that affects expression of the ChroM1 chromatin remodeling protein. This method involves incubating a human cell, which expresses the ChroM1 polypeptide of SEQ ID NO:2, in the presence or absence of a potential enhancer or inhibitor compound that affects expression of ChroM1. The potential enhancer or inhibitor compound screened may be screened from among the compounds in chemical libraries such as are available at many pharmaceutical companies. After incubation, the level of expression of ChroM1 in the presence of the potential enhancer or inhibitor compound is determined relative to the level of expression of ChroM1 in the absence of the potential enhancer or inhibitor compound. A screened potential enhancer or inhibitor compound is identified as an enhancer compound if the level of expression of ChroM1 in the presence of the potential enhancer compound is substantially more than that in the absence of the potential enhancer compound. Conversely, a screened potential enhancer or inhibitor compound is identified as an inhibitor compound if the level of expression of ChroM1 in the presence of the potential inhibitor compound is substantially less than that in the absence of the potential inhibitor compound. The identified enhancer or inhibitor compound can further be isolated once it is identified.

While a compound that ehances the expression of ChroM1 can be used to treat osteoporosis, a compound that inhibits the expression of ChroM1, such as identified by the method described above can be used, for instance, to treat osteopetrosis or osteoscarcoma by administering the inhibitor compound to a patient in need thereof.

As shown in the Example, ChroM1 was demonstrated to bind promoters of genes that play an important role in bone biology. This provides for directly manipulating the activity of these genes by using a compound that affects the binding of ChroM1 to the promoters of these genes, which may result in a restricted and directed effect on key bone proteins in a tissue and time-specific manner. A number of genes that play an important role in bone biology, i.e., osteoblast cell function and differentiation, and their promoters have been identified in the prior art. The laboratory of the present inventors have used the promoters of three genes, estrogen receptor α (ERα; Genbank accession no. X63118), bone morphogenic protein-4 (BMP-4; Genbank accession no. U43842; van de Wijngaard et al., 2000), and osteocalcin (Lian et al., 1998) for binding studies with ChroM1. The studies of ChroM1 binding to promoters as disclosed in the Example show that in a chromatin immunoprecipitation (ChIP) assay, the DNA binding domain (DBD) of ChroM1 binds to the A/T-rich distal promoter region of ERα and BMP-4. ChroM1 binding was reduced in the presence of distamycin A, an antibiotic that blocks binding to A/T-rich regions.

In another aspect of the present invention, a method of identifying A/T-rich promoter regions of genes involved in modulation of osteoblast differentiation and which are capable of binding to the DNA binding domain of ChroM1 is provided. This method can be used to identify a promoter of a known gene or of an unknown gene. For instance, a microarray of human genomic DNA fragments, as is believed to be commercially available and is believed to be within the skill of those in the art to produce, can be contacted with a peptide containing the DNA binding domain of ChroM1 to identify A/T rich promoter regions of potential genes involved in modulation of osteoblast-differentiation. The method involves contacting fragments of genomic DNA with a peptide containing a DNA binding domain comprising residues 2429-2437, preferably comprising residues 2333-2480, of the ChroM1 protein of SEQ ID NO:2 and identifying a fragment of genomic DNA bound by this peptide. The nucleotide sequence of the promoter region on the fragment bound by the peptide is determined so that the A/T-rich promoter region of a gene involved in modulation of osteoblast differentiation is identified.

ChroM1 is believed to be a chromatin remodeling protein that opens up chromatin to allow access to a promoter region where the DNA binding domain (DBD) of the ChroM1 protein can bind an AT-rich promoter region of a gene involved in osteoblast differentiation (from osteogenic progenitor cells to osteoblasts and muscle cells) in the presence of a modulator compound, i.e., estradiol for estrogen receptor promoter, to initiate transcription. Thus, ChroM1 functions in transcriptional regulation of genes involved in osteoblast differentiation in the presence of a modulator that can be screened from chemical libraries, such as libraries of pharmacological compounds and metabolites.

The present invention further provides for a method of screening for and identifying a compound which stimulate differentiation of osteogenic progenitor cells by using chromatin immunoprecipitation (ChIP) with ChroM1 and anti-ChroM1 antibodies followed by analysis of PCR amplification products of the DNA from the immunoprecipitated chromatin. The general ChIP method is disclosed in Orlando (2000) and Chen et al (1999). The present method using ChIP involves incubating human cells, which express the ChroM1 chromatin remodeling protein of SEQ ID NO:2 with a potential stimulator compound and adding formaldehyde to the incubated human cells to crosslink proteins to DNA in chromatin by in vivo fixation. Once the proteins are crosslinked to DNA in chromatin, the crosslinked chromatin is sonicated to solubilize the crosslinked chromatin. The solubilized crossliked chromatin is then immunoprecipitated with antibodies specific for the ChroM1 protein of SEQ ID NO:2 to form immunocomplexes. DNA from the immunocomplexes are recovered and incubated under amplification conditions with oligonucleotide primers capable of amplifying an A/T-rich promoter region of a gene involved in control of osteoblast cell differentiation if present in the recovered DNA. For example, oligonucleotide nucleotide primers Erp3 (SEQ ID NO:28) and Erp4 (SEQ ID NO:29) were used in experiments in the Example to amplify a 204 bp ERα A/T-rich promoter region (SEQ ID NO:32) from the DNA recovered from ChIP. Likewise, oligonucleotide primers BMPpr3 (SEQ ID NO:20) and BMPpr4 (SEQ ID NO:21) were used in the experiments in the Example to amplify a 145 bp BMP-4 A/T-rich promoter region (SEQ ID NO:33) from the DNA recovered from ChIP. If an amplication product is detected which corresponds to the A/T rich promoter region capable of being amplified with the oligonucleotide primer used, then the potential stimulator compound is identified as a stimulator compound. Besides A/T-rich promoter regions of human estrogen receptors (i.e., ERα), human bone morphogenic protein (i.e., BMP-4), or osteocalcin, promoters of other known genes involved in osteoblast cell function and differentiation and promoters identified in the method of identifying A/T-rich promoter regions according to the present invention can be used with the appropriate oligonucleotide primers once the nucleotide sequences of the A/T-rich promoter regions are known.

Having now generally described the invention, the same will be more readily understood through reference to the following examples which are provided by way-of illustration and is not intended to be limiting of the present invention.

### EXAMPLE

The laboratory of the present inventors has cloned and identified a gene of interest, designated ChroM1, that is expressed by pre-osteoblastic cells. The comparison of this gene to the genome database with a variety of algorithms, combined with dry and wet biology, deepens the knowledge on the structure and function of the genes. The ChroM gene was mapped on human chromosome 16 and the genomic structure with intron-exon boundaries that include the full-length gene structure and alternative splicing forms was determined. The protein is involved with genome integrity and the control of chromatin-remodeling complexes that facilitate gene expression by helping transcription factors gain access to their targets in chromatin. ChroM is related to the SWI/SNF family members from yeast but it is distinguished from these family members because it contains a chromodomain and possesses regions that link the protein to the cell membrane. In this example, evidence is presented that the identified human unique ChroM harbors structural motifs at the N-terminus of chromo domain followed by a SNF2-like domain that is characteristic of AAA-ATPases, DEXDc-helicases super family, and TCH region preference for A/T rich regions. These motifs have homology to functional proteins that control cell growth. ChroM is expressed in proliferating cells and is correlated with the G1 phase. The protein contains a signature motif LXXLL (NR box) that is necessary and sufficient to permit interaction with nuclear receptors.

Immunohistochemistry and FACS studies revealed that ChroM1 localizes at the cell membrane and cytoplasm of osteoprogenitor cells and the protein is translocated to the nucleus for its activity and is enriched in the nucleus of cancer cells such as osteosarcoma. The data presented below suggest that ChroM represent a novel family of membrane factors that act also in chromatin-remodeling complexes. No other protein resembling this family has yet been identified. This unique protein however has some conserved functional regions from yeast to human.

Material and Methods

In vitro culture: Human bone marrow stromal cells (MSC) were collected from surgical aspirates of bone marrow (normal donors male and female at age 2.7 to 49 years) to prepare ex vivo culture plated at low-density (1.5×10⁴ cells/cm²). Clonal populations of human bone marrow stromal cells were obtained from the single cell derived cells (previously described in Shur et al. 2000). Human trabecular bone cultures (TBC) were established from fresh trabecular bone explants from femoral heads during orthopaedic procedures. Bone samples obtained from 14 donors aged 60-80 years were cultured. Primary marrow stromal cells (MSC) and tranbecularbone cells (TBC) were cultured in Dulbecco's Modified Essential Medium (DMEM) with the addition of 10% heat-inactivated fetal calf serum (FCS). The growth medium for clonal populations of MSC was supplemented with 10⁸M dexamethasone (Dex) (Ikapharm, Israel) and 10⁻⁴M L-ascorbic acid phosphate magnesium salt (Sigma, Israel).

Gene expression analysis of MSC: Total RNA was extracted from cultured cells (EZ RNA kit, Biological industries, Bet-Haemek, Israel). The RNA was reverse transcribed using avian myeloblastosis virus reverse transcriptase (AMV-RT) and oligo-dT, in order to generate cDNA. This cDNA then served as a template for the polymerase chain reaction (PCR) (Takara Shuzo Co. Ltd., Japan). The integrity of the RNA, the efficiency of RT reaction and the quality of cDNA subjected to the RT-PCR were taken into account by controls in which a transcript of Glucose-3-Phosphate Dehydrogenase (G3PDH) was amplified using the primers, G3PDHf: ACCACAGTCCATGCCATCAC (SEQ ID NO:9)and G3PDHr TCCACCACCCTGTTGCTGTA (SEQ ID NO:10), obtained from Clontech, Palo Alto, Calif. Genomic DNA contamination was excluded by using primers for c-Myc and TGFb1 that amplified products of different size in the genome when compared to cDNA. The same cDNA derived from each donor and/or clone was used for PCR analysis with specific primers ChroMf: AGCAACACAGATGTC (SEQ ID NO:11), ChroMr: ATCAGGAATTCCTTGAGGTTG (SEQ ID NO:12).The reaction products were separated by electrophoresis in 1% agarose gels (SeaKem GTG, FMC, USA) in Tris Borate EDTA (TBE) buffer. The amplified DNA fragments were stained by ethidium bromide measured for optical density (OD) by densitometry (Bio Imaging System, BIS 202D) and analyzed using “TINA” software. PCR amplification was performed at least twice and subjected to semi-quantitative analyses by comparison of the OD of PCR products for ChroM normalized to the OD of co-amplified G3PDH-PCR product.

Immunohistochemistry: The specimens were fixed in 4% formalin in PBS (phosphate buffered saline) immediately after excision of embryos or organ. Where needed, decalcification was performed with 10% EDTA in PBS (pH 7.4) in 4° C. for 2 days. The specimens were washed in PBS and embedded in paraffin (50-60° C.) and cut serially in 5 widths.

For staining, deparaffinization in xylene for 15 minutes and rehydration by an alcohol gradient followed with PBS washing. For immunohistochemistry, the section was blocked with 10% normal goat serum in 1% BSA/PBS for 30 minutes. Immunohistochemical stains used for localizing various proteins on tissue from embryogenic stages of development-in adult mice and in section from human bone. The immuno-detection used anti-ChroM IgG-purified antibody from rabbit serum. The signal was amplified with second antibody, goat anti-rabbit-biotin (Dako) and extravidine-peroxidase (Sigma). The reaction was detected with the chromagen, DAB (DAB-3,3′-Diaminobenzidine tetrahydrochloride, Sigma) that creates a hard dissolving salt sedimentation after reacting with the peroxidase. For counterstain, Hematoxilin for cell nuclei staining was used.

Flow Cytometric Analysis: Cells were EDTA-released from cultures. Fluorescence Activated Cell Sorting (FACS) analyzed the single cell suspension. The staining for surface antigens employed biotin conjugated antibodies to CD-44, CD-51, CD-61, CD62P, CD62L, CD62E (Pharmingen). Cell cycle markers were analyzed with antibodies: KI-67 (Dako), cFOS, cJUN (Oncogene) and Cyclin B1 and Cyclin Dl using intracellular staining procedure. Negative control for intracellular staining employed the isotype-matched IgG (Mouse IgG1 and Rabbit IgG) at the same concentration as the antibody of interest. Secondary antibodies used were Extravidine-PE (Sigma) or FITC-conjugated-anti-rabbit (Jackson immune Research Laboratories). FACS staining was performed according to technical protocols (website www.pharmingen.com). Cell Cycle was also analyzed by Propidium Iodine (PI). For each antibody, 1×10⁶ cells was used for the staining procedure. Finally, 1×10⁴ cells were quantified and the analysis was performed using software from Becton Dickinson.

Protein analysis, cell biotinylation, immunoprecipitation (IP) procedure, SDS-PAGE gel and Western blot analysis: These procedures and analyses were performed according the standard protocols website www.protocol-online.net/Protocol.htm. Briefly, immunoprecipitation was performed with ChroM1 antibody (dilution 1:700) (6 mg/ml) incubated overnight with Protein A-Sepharose beads (Sigma). The immuno-complexes were separated on 6.5% SDS-PAGE gel for 2 hrs, then transferred for 30 min to the nitrocellulose blots and probed with primary antibody diluted 1:1000 for 1 hour, followed with secondary antibody goat anti-rabbit-biotin IgG (1:2000) and Extravidin Peroxidase (1:4000) for detection with chemiluminescent substrate (Pierce).

Chromatin immunoprecipitation (ChiP): This technique offers the ability to detect protein that binds to protein complexes bound to DNA. The technique is based on formaldehyde fixation to chromatin (Orlando, 2000 and Chen et al., 1999).

ATPase activity: IP protein used to determine phosphate ions released during ATPase reaction when incubated at 37° C. The protein activity was determined in the presence or absence of DNA primer, GCGCAATTGCGCTCGACGATTTTTTAGCGCAATTGCGC (SEQ ID NO:13), that form a stem loop structure (Bayko et al., 1988 and Muthuswami et al., 2000).

Electrophoretic mobility shift assay (EMSA): EMSA was performed between recombinant protein (rP) containing the DNA binding domain (residues 2333-2480 from sequence of ChroM1) and ³²P-labeled 32-mer oligonucleotide containing 24 A·T nucleotides, GATCCATATATATATATATATATATATATGCA (SEQ ID NO:14). 1-3 μg of rP and 100,000 cpm of ³²P-labeled oligonucleotides were used for the binding reaction in the presence or absence of 100 fold excess cold oligonucleotide. The reaction was carried out in 20 μl of binding buffer (26 mM Hepes, pH 7.9, 2 mM MgCl₂, 40 mM KC1, 1 mM dithiothreitol and 0.25% BSA). The samples were incubated for 15 min at room temperature prior to loading in 5% native polyacrylamide gel in 0.25 Tris borate-EDTA (TBE). The gel was run for 40 min at RT and in TBE buffer. The gels were then dried and autoradiographed.

Chromosome localization: In the analysis for chromosomal gene localization, the Stanford Human Genome center radiation hybrid (RH) panel was used. This kit provides high-resolution maps of the human genome using a somatic-cell approach. Hamster cells containing a human chromosome are blasted with radiation to scramble the DNA. The damaged cell is then fused with a non-irradiated hamster cell and grown into a colony of hybrids. DNA isolated from these radiation hybrid cells provides the background used to order STSs and to determine the distance between them. The distances are calculated using statistical analyses. The Stanford G3 maps were constructed using a panel of 83 whole genome radiation hybrids (the Stanford G3 panel) and 10,478 sequence tagged sites. The sites were derived from random genomic DNA sequences, previously mapped genetic markers and expressed sequences. These maps cover the majority of the human genome, with the markers lying at an average distance of 500 kb apart.

Statistical analysis—Statistical analysis of differential gene expression by RT-PCR of cultured cells derived from various donors was performed by ANOVA test. Results were considered significant for P<0.05.

Softwares used for bioinformatic analysis: cDNA translation to protein was analyzed using BLASTN from NCBI, “Smart” program (website www.smart.embl-heidelberg.de)

| Web site | Goal | Remarks |
|---|---|---|
| www.expasy.ch/tools/ | Translation, ORF | |
| www.expasy.ch/cgi-bin/protparam | Protein parameters | |
| Dot.imgen.bcm.tmc.edu:9331/seq-util/seq-util.html | Changing format | |
| Psort.nibb.ac.jp/form2.html | Subcellular localization | |
| Coot.embl-heidelberg.de/SMART | Functional and structural domains | |
| www2.ebi.ac.uk/ppsearch/ | Motifs | with graphics |
| www.cbs.dtu.dk/services/NetOGlyc/ | O-glycosylation | |
| azusa.proteome.bio.tuat.ac.jp/sosui/submit.html | Transmembrane domains | solubility |
| www.ch.embnet.org/software/TMPRED_form.html | | |
| www.biokemi.su.se/˜server/toppred2/toppredServer.cgi | | |
| www.at.embnet.org/embnet/tools/bio/PESTfind/ | PEST | |

Protein analysis: web sites and databases.

| Web site | Application |
|---|---|
| www.expasy.ch/tools/dna.html | Translation, ORF |
| www.expasy.ch/cgi-bin/protparam | Protein parameters |
| www.psort.nibb.ac.jp/form2.html | Subcellular localization |
| coot.embl-heidelberg.de/SMART | Functional and structural domains |
| www2.ebi.ac.uk/ppsearch/ | Motifs |
| www.pbil.ibcp.fr/cgi-bin/npsa_automat.pl?page=npsa_prosite.html | |
| www.cbs.dtu.dk/databases/PhosphoBase/predict/predform.html | Phosphorylation |
| www.cbs.dtu.dk/services/NetOGlyc/ | O-glycosylation |
| www.ch.embnet.org/software/TMPRED_form.html | Transmembrane domains |
| www.biokemi.su.se/˜server/toppred2/toppredServer.cgi | |
| 130.237.130.31/tmap/single.html | |
| www.at.embnet.org/embnet/tools/bio/PESTfind/ | PEST |

PROSITE is a database of protein families and domains. It has biologically significant sites, patterns and profiles that help to reliably identify to which known protein family (if any) a new sequence belongs.

ProDom database has been designed as a tool to help analyze domain arrangements of proteins and protein families.

Pfam is a collection of protein families and domains. Pfam contains multiple protein alignments and profile-HMMs of these families. Pfam is a semi-automatic protein family database, which aims to be comprehensive as well as accurate.

Blocks are multiply aligned ungapped segments corresponding to the most highly conserved regions of proteins. “Block Searcher”, “Get Blocks” and “Block Maker” are aids to detection and verification of protein sequence homology. They compare a protein or DNA sequence to a database of protein blocks (current version), retrieve blocks, and create new blocks, respectively.

PRINTS is a compendium of protein fingerprints. A fingerprint is a group of conserved motifs used to characterize a protein family; its diagnostic power is refined by iterative scanning of OWL. Usually the motifs do not overlap, but are separated along a sequence, though they may be contiguous in 3D-space. Fingerprints can encode protein folds and functionalities more flexibly and powerfully than can single motifs: the database thus provides a useful adjunct to PROSITE.

Results and Discussion

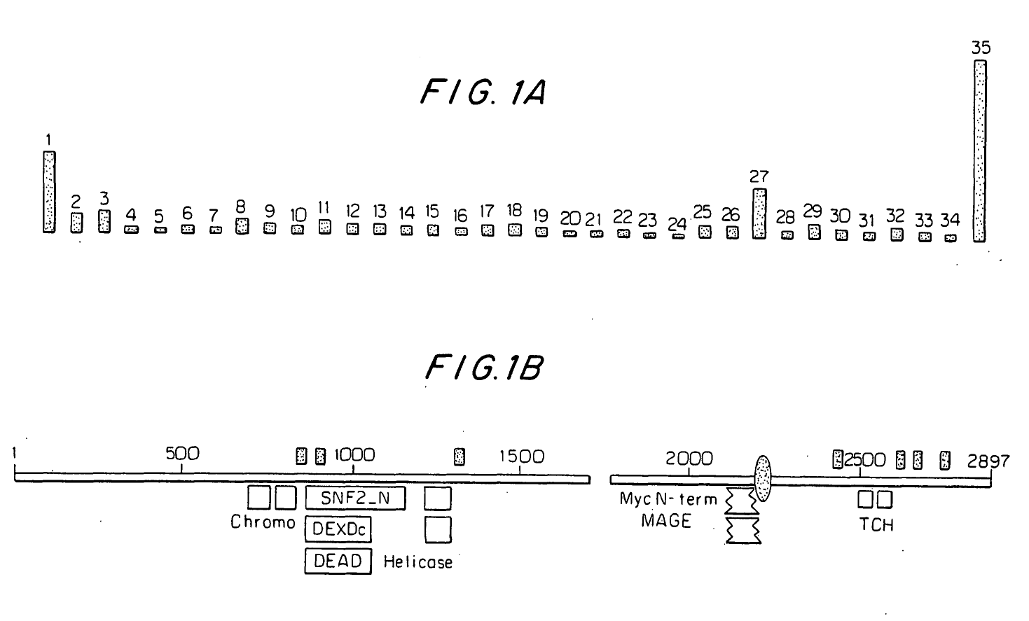

A cDNA which encodes for the full length cDNA ChrOM1 extends 11337 nucleotides. The chromosomal localization was analyzed using radiation hybrid panel RH-G3 (Stanford Human Genome center). The gene was mapped to chromosome 16q12.2 based on the genomic marker GDB locus D16S3137 SHGC AFMa061lyb5 with LOD 6.4, targets YACs (802h4, 813e2, 922f1, 959g5). PCR analysis verified the amplified product on these YACs. The localization was confirmed with the advancement of the human genome project and localized to the contigs AC007345, AC007906. Bioinformatic analysis of full-length cDNA with 35 exons that represent their size and organization (FIG. 1A). The largest open reading frame (ORF) of the cDNA result with 8694 bp, where the first stop codon in frame appears in the last exon of 3431 bp which is translated only in the first 877 bp and continues with 3′ UTR sequences and the poly (A) tail. The cDNA ORF translation results in a protein of 2898 amino acids from the first methionine with the predicted protein being approximately 326 kDa in molecular weight. Various softwares for proteins were used to localize the ChroM1 protein to different cellular compartments (membranous, cytoplasmic and nuclear). Examples of other proteins with dual localization are presented in Table 1.

TABLE 1
Proteins shuttle and expression of dual localization (transmembrane and nucleus)

| | Mechanism of | | |
|---|---|---|---|
| | intra-cellular | | |
| Protein | translocation | References | Function |
| BAA24799 | Unknown | Yang L et al, 2000 | Focal adhesion protein demonstrated |
| Hic-5 | | | to be associated with nuclear matrix |
| | | | and function as steroid receptor coactivators |
| AAF68983 | Unknown | Nakamura T et al, 2000 | Dual localization in nuclear speckles |
| HuASH1 | | | and tight junctions, a member of |
| | | | trithorax group of genes with chromatin |
| | | | remodeling activity |
| NP_002678 | Unknown | Ouyang P. | differentiation-specific desmosomal |
| Pinin/Mema/DRS | | | protein, nuclear phosphoprotein |
| XP_030094 | Unknown | Gottardi C J et al, 1996 | Tight-junction associated member of the |
| ZO-1 | | | family of Guanilate-cyclase homologues, |
| | | | nuclear localization is associated with |
| | | | proliferative, immature status |
| AAG33848 | Proteolytic cleavage | Artavanis-Tsakonas S, | Transmembrane receptor, intracellular |
| Notch | | et al, 1995 | domain translocates to the nucleus and |
| | | | forms a complex with transcriptional |
| | | | repressor |
| CIZ AB019281 | | Nakamoto T et al., 2000 | Focal adhesion and nucleus |
| EGF receptor | | Ref | Transmembrane receptor translocates to |
| | | | the nucleus |

For ChroM1 it was suggested that the protein possesses transmembrane (TM) domains and is defined also as a nuclear protein with 69.6% probability by PROST analysis. The potential of the protein to translocate to the nucleus is supported by the existence of a consensus sequence bipartite nuclear localization signal, nuclear functional domains, chromo domains, SNF2, Helicase-C and SANT (Table 2). CHROMO (Chromatin Organization Modifier) is a conserved region involved with remodeling of chromatin (FIG. 1B, Table 2). It has been hypothesized that the chromo domain may be a vehicle that delivers both positive and negative transcription regulators to the sites of their action on chromatin. Proteins that contain a chromo domain appear to fall into three classes. The first class includes proteins having an N-terminal.chromo domain and chromo shadow, mammalian modifier 1 and modifier 2. The second class includes proteins with single a chromo domain, such as the Drosophilae protein Polycomb (Pc). In the third class, paired tandem chromo domains are found in mammalian DNA-binding/helicase proteins CHD-1 to CHD-4 and yeast protein CHD1.

The existence of two chromodomains followed by DEAD/SNF2 helicase C domains suggests that ChroM1 is involved in chromatin regulation. Proteins that contain a N-terminal SNF2 domain appear to be distantly related to the DEAD box helicases however no helicase activity has ever been demonstrated for these proteins. ChroM1 has similarity to the SNF2 subgroup and contains motifs of chromo domain genes; the extended sequence analysis shows that it belongs to an entirely novel protein family.

Regions with similarity to DEAD-like and helicase DNA binding protein are well conserved through evolution and are expressed by a series of viruses and bacteria as transmembrane proteins and in some hypothetical proteins. This domain is found in proteins involved in a variety of processes including transcription regulation (e.g., SNF2, Brahma), DNA repair (e.g., RAD16, RAD5), and chromatin unwinding (e.g., ISWI). SNF2-domain is recognized also in BRG-1 (human), Brahma BRM (Gallous), kismet (Drosophilae), or SWI/SNF related (Yeast).

TABLE 2
Nuclear domains of ChrOM1

| Domain | Localization | Program |
|---|---|---|
| Chromo domain | 688-754; | Prosite, SMART |
| | 771-829 | |
| SNF2 | 863-1150 | Pfam A |
| helicase motif I | 880-892 | |
| helicase motif Ia | 912-920 | |
| helicase motif III | 1023-1030 | |
| helicase motif IV | 1076-1086 | |
| helicase motif V | 1258-1269 | |
| helicase motif VI | 1287-1297 | |
| helicase C domain | 1212-1296 | Prosite, SMART |
| SANT | 1834-1893 | SMART, |
| KR-like | 2429-2436 | Bourachot et al, 1999 |
| bipartite NLS | 565-585 | PSORT |
| NLS | 1448-1455 | |
| | 1478-1485 | |
| | 1577/9-1584/6 | |
| | 2116-2123 | |
| | 2426-2433 | |
| | 2504-2511 | |
| | 2537-2542 | |
| | 2632-2637 | |

A SANT-like domain is involved in protein-protein or protein-DNA interactions. The SANT-like domain following the SNF2 distinguishes the ChroM protein from the third class of chromodomain proteins. The presence of the SANT domain following Chromo and the presence of the SNF2 and helicase domains conserved through evolution with other proteins, including the hypothetical protein T04D1.4 from C. elegans, the kismet long isoform of Drosophila and several human hypothetical proteins. It also appears separately at the Phylogenetic tree based on the BLASTp algorithm and multiple alignments to construct a new family that includes ChrOM, AL031667 (human C-Helicase), kismet long isoform (Q9NI64), and T33152 (C. elegans hypothetical protein T04D1.4).

In addition, several domains of unknown function that are shared between CHROMO-domain/SNF2 family members were identified, such as CR1-CR3, BRK/TCH and DUF94 (Table 3).

TABLE 3
Domains of unknown function in ChroM1 protein

| Domain | Localization | Program |
|---|---|---|
| DUF94 | 1194-1306 | SMART |
| CR1 | 1492-1559 | Therrien et al., 2000 |
| CR2 | 1616-1706 | |
| CR3 | 1838-1990 | |
| TCH/BRK | 2482-2531 | SMART |
| | 2556-2600 | |

The ChroM1 protein sequence contains a high percentage (10.5%) of serine amino acid residues which makes the protein acidic. Specifically in exon 27, an unusually high number of serine amino acid residues that form a 64-amino acid stretch of residues was identified. Database analysis revealed additional proteins which contain a stretch of serine residues and are recognized as nuclear phospho-proteins that form multi-protein complexes and participate in transcriptional activation (Table 4). These proteins are also known to participate in tumorigenic processes. Additionally, the N-terminus of exon 27 contains two conserved domains: a Myc N-terminal domain of an oncoprotein known to be involved in cell replication and a MAGE domain expressed in a wide variety of tumors (Table 5).

TABLE 4
protein with a stretch of serine residues

| | Serine | |
|---|---|---|
| Protein | stretch* | Function |
| NP_004732 | 15aa -87% | May play an important role in |
| Nucleolar | 132aa -79% | transcription catalyzed by RNA |
| phospho- | | polymerase I |
| protein | | |
| p130 | | |
| NP_057417 | 36aa -89% | Splicing co-activator, RNA binding |
| SRm300 | 41aa -100% | |
| P42568 | 42aa -100% | Transcriptional activator resulted |
| myeloid/ | | from chromosomal rearrangement, |
| lymphoid | | proto-oncogene |
| Or mixed- | | |
| lineage | | |
| leukemia | | |
| NP_002678 | 61aa -77% | Differentiation-specific desmosomal |
| Pinin/Mema/ | | protein, Nuclear phosphoprotein |
| DRS | | Pinin was demonstrated in cell |
| | | membrane and Desmosomes/DRS was |
| | | demonstrated in the nucleus |
| BAA24570 | 44aa -84% | modulating nucleosome structure and |
| MB20 | | gene |
| | | expression during brain development |
| ChrOM1 | 66aa- 78% | |

*displays percent of serine amino acid in the stretch

TABLE 5
Motifs associated with oncoproteins

| Domain | Description | ChrOM Sequence |
|---|---|---|
| Myc-N-term | Myc proto-oncogene | 2106-2207 |
| MAGE | Melanoma antigene encoding gene | 2125-2202 |

The ChroM1 protein is suggested by the inventors to play a role in the assembly and activity of the nuclear receptors (NR) transcription complex. The NR box is characterized by a LXXLL sequence flanked with a short stretch of amino-and carboxyl-terminal amino acids and is both necessary and sufficient for ligand-dependent interactions of protein complexes with AF2 domains of nuclear receptors. In addition, the BRK motifs that were identified in other proteins (Table 3) and that were recognized for their interactions with estrogen or glucocorticoid receptors, such as BRG1 and BRM, strengthen their function in the regulation of steroid receptors activity.

The ChroM1 protein is believed to be an integral membrane protein associated with other proteins (www.softberry.com/protein.html), has several transmembrane domains and contains a GXXXG motif that is important in protein interactions. Transmembrane domains and motifs of interactions with extracellular or intracellular proteins are summarized in (Table 6).

TABLE 6
Transmembrane domains and motifs of interaction
with extracellular and cytoskeletal proteins

| | |
|---|---|
| Localization | Program |

A. Transmembrane

| | | |
|---|---|---|
| Trans Membrane (TM) | 902-923 | TMPRED, toppred, |
| | | TMAP |
| | 969-989 | |
| | 1252-1276 | |
| | 2454-2474 | |
| | 2640-2660 | |
| | 2682-2702 | |
| | 2797-2820 | |
| GxxxG | 2693-2697 | |

B. Interaction with Extracellular

| | | |
|---|---|---|
| RGD cell attachment | 1544-1546 | Prosite |
| LDV | 2550-2552 | |
| LRE | 2386-2388 | |
| Kringle | 2543-2548 | Prosite |

C. Interaction with Cytoskeleton

| | | |
|---|---|---|
| Caldesmon | 499-670 | BLASTp-CD |
| Duplin | 512-791 | BLASTp |
| (b-catenin interacting protein) | | |
| Actin | 1006-1057 | BLOCKs |
| F-actin capping protein A subunit | 2556-2594 | BLOCKs |
| Tropomyosin | 1430-1484 | BLOCKs |
| | 2035-2071 | |

The ChroM1 protein is associated with other proteins through cell attachment sites and cytoskeletal components (Table 6). “Blocks” that suggest interaction with actin, F actin and thropomyosin are present. At the N-terminus, it is suggested that the ChroM1 protein binds to caldesmon and duplin b-catenin interacting protein. Caldesmon functions in actin and myosin binding and is implicated in the regulation of actomyosin interactions that stimulate actin binding of tropomyosin, which binding increases the stabilization of actin filament structure. Caldesmon plays an essential role during cellular mitosis and receptor capping. Phosphorylation causes caldesmon to dissociate from microfilaments and reduces caldesmon binding to actin, myosin, and calmodulin.

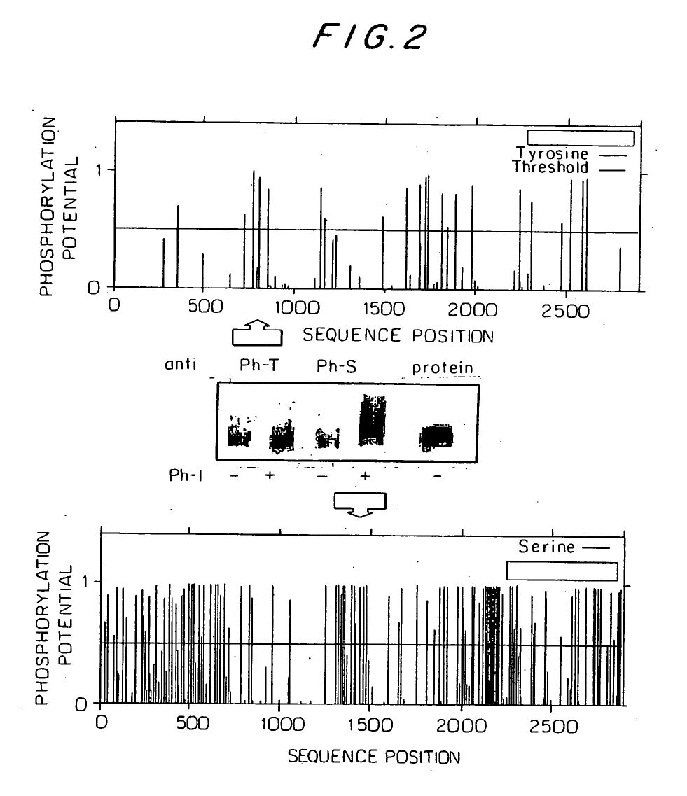

Post-translational modifications: ChroM1 is predicted to be a highly phosphorylated protein (FIGS. 2A-2C) and it demonstrated phosphorylation of serine and tyrosine residues. It is also a substrate for phosphorylation by protein kinase C (35 sites), protein kinase A (40 sites), Casein kinase II (62 sites), Tyrosine kinase (4 sites) and cAMP/cGMP-dependent protein kinase (6 sites), CaMII calmodulin dependent kinase II (26 sites), and p34cdc2 (1 site). Also predicted for multiple O/N glycosylation sites, amidation and myristoylation.

Chromatin remodeling factors participate in transcriptional activation by nuclear receptors. The presence of seven LXXLL nuclear receptor activation boxes in the ChroM1 sequence (Table 7) suggests that it is binding to the nuclear receptors. Bioinformatic analysis revealed homology between ChrOM and members of the SWI/SNF2 complex that were shown to interact with estrogen and glucocorticoid receptors (Fryer et al., 1998; Ichinose et al., 1997 and Muchardt et al., 1993). The SNF2 mammalian homology is BRM and BRG that possess SNF2 and Helicase C domain. KR is homologous to HMGI/Y DNA binding domain and was shown to coordinate the activity of mammalian brm/SNF2alpha (Bourachot et al., 1999). BRK/TCH is a domain of unknown function that is shared between SWI/SNF2 and a class of chromodomain proteins and that is included in ChrOM1 (Elfring et al., 1998; Daubresse et al., 1999).

TABLE 7
Nuclear receptor (NR) interaction domains

| Domain | Localization | Program |
|---|---|---|
| LxxLL box | 386-390 | SMART |
| | 868-872 | |
| | 1036-1040 | |
| | 2031-2035 | |
| | 2659-2663 | |
| | 2721-2725 | |
| | 2793-2797 | |
| KR-like motif | 2429-2436 | Bourachot et al. 1999 |
| BRK/TCH | 2482-2531 | Elfring et al., 1998 |
| | 2556-2600 | Daubresse et al., 1999 |

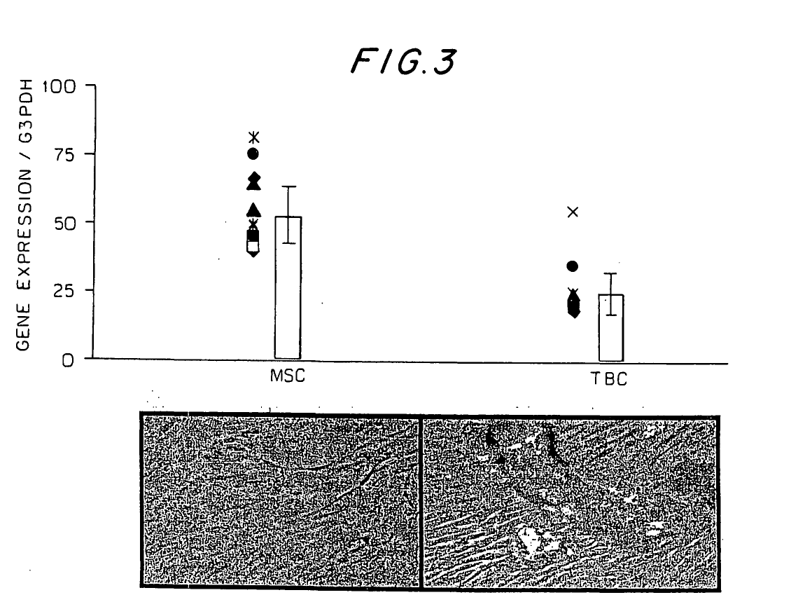

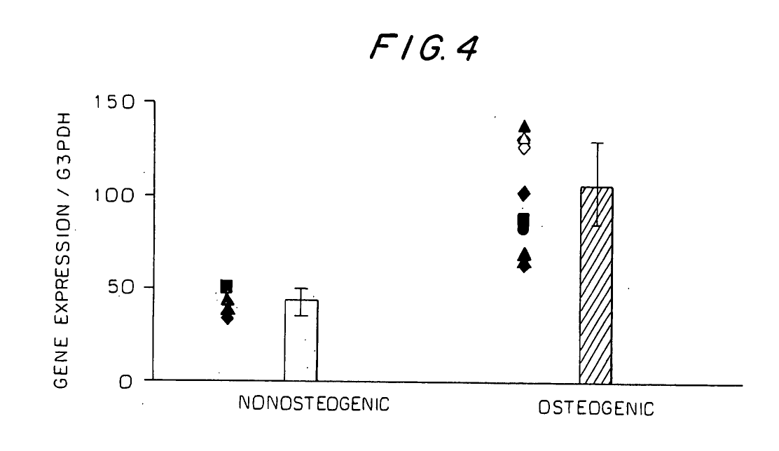

The cDNA size was confirmed by Northern blot analysis (data not shown) of RNA extracted from cells representing two model systems of cultured osteoblasts (MSC and TBC; FIG. 3). The RNA was further analyzed to quantify the level of expression of mRNA for ChroM1. MSC and TBC used for RNA extraction from each donor was reverse transcribed to cDNA. Each cDNA, obtained from equal amounts of total RNA, was analyzed for ChroM expression and compared to equal amounts of G3PDH-PCR that served as a baseline for semi-quantitative analysis. Messenger RNA for ChroM1 was expressed 2.4-fold higher in MSC than in TBC (p<0.0001, FIG. 3). These RNA samples had been previously studied and genomic DNA contamination was excluded. MSC is a heterogeneous cell population representing cells at different stages of differentiation. Thus, the cloning of cells and a comparison for osteogenic potential between cells that possess osteogenic or nonosteogenic capacity was studied when implanted subcutaneously in mice. ChroM1 expression was 2.5-fold higher in osteogenic than in nonosteogenic clones (FIG. 4, p<0.002).

Antibodies were generated based on the protein sequence and were used for protein expression analysis. At the tissue level, paraffin sections were analyzed to identify the cells expressing the ChroM1 in bone sections (FIG. 5). Immuno-staining demonstrates that positively stained osteoprogenitors are located immediately adjacent to the mature osteoblasts (FIG. 5, osteoblast, arrow head). At the bone marrow, unstained cells were counter stained with methylene green.

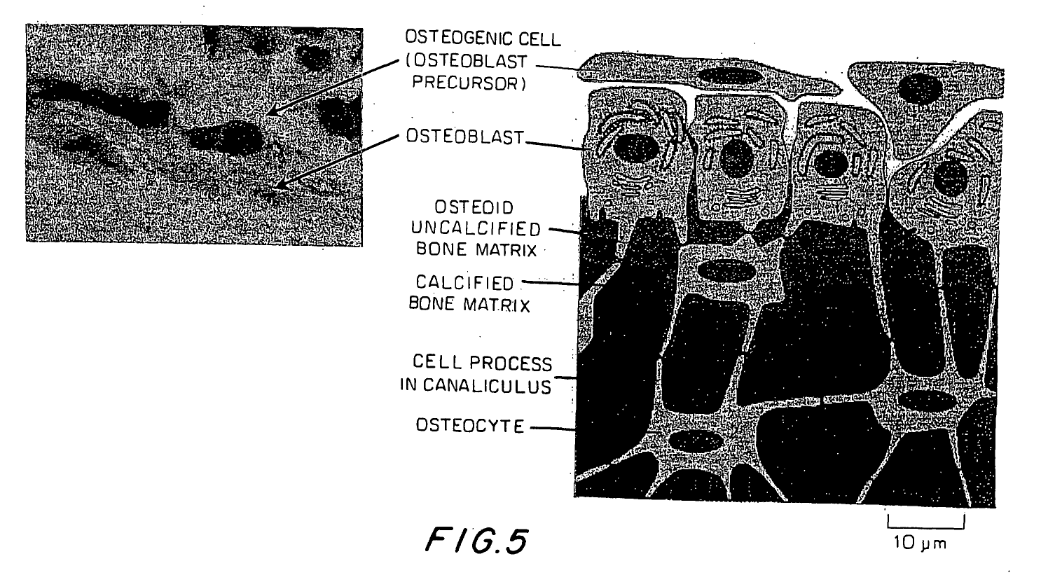

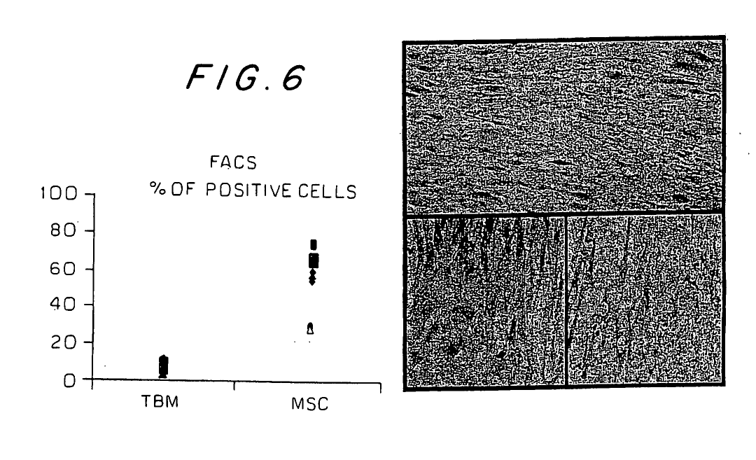

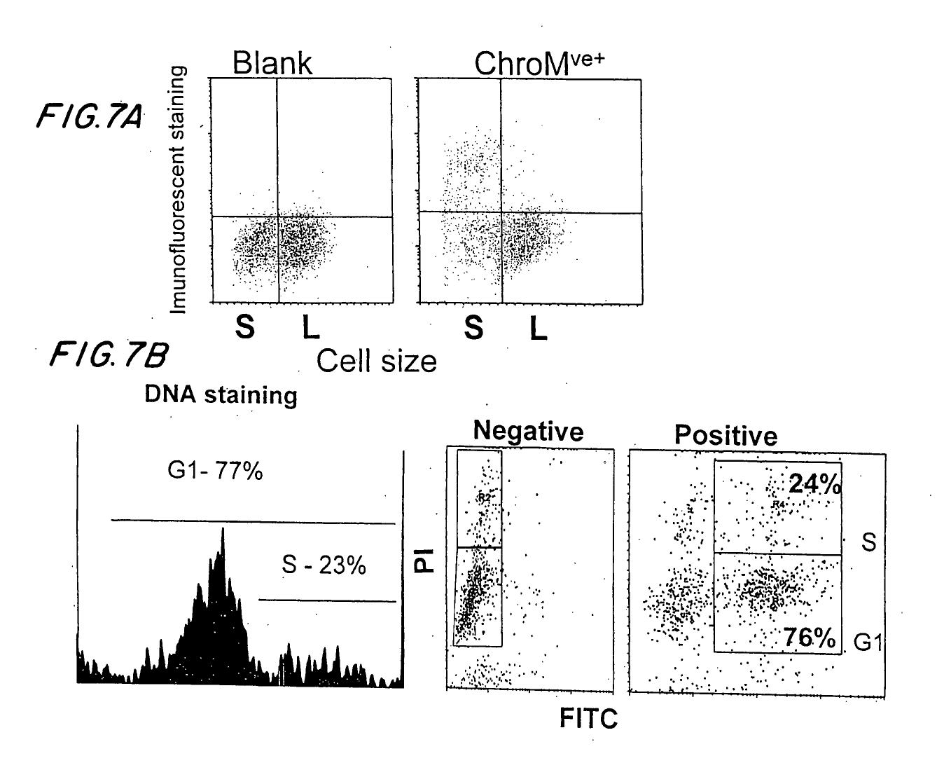

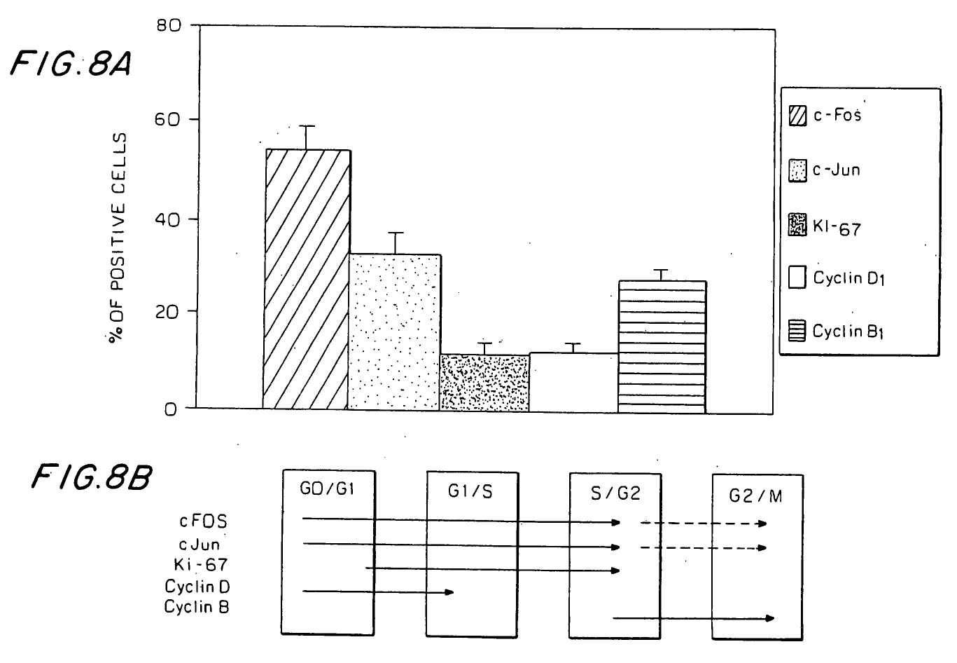

Freshly isolated bone marrow cells (BMC) were also cultured and an adherent fibroblast-like layer of enriched marrow stromal cell (MSC) population was formed. These two cell populations were analyzed by FACS for surface antigen expression and the results show that 8%±3 ChroM1^{VE+} cells from BMC (FIG. 6) and 55%±18 of MSC from various donors were positively stained, which is a 7-fold enrichment of ChroM1^{VE+} cells in the MSC than in BMC (FIG. 6; p>0.0012). These cells were also immunostained, and expressed membrane and cytoplasm staining. Further analysis on the MSC and measurements of cells estimated the cell size of ChroM1^{VE+} to correspond to >80% of ChroM1^{VE+} cells with a small cells size (less then 30% of maximal cell size) (FIG. 7A). The positive cells were estimated for relative size and cytoplasm granulation was correlated to small cells having high proliferating capacity (FIG. 7B). ChroM1^{VE+} were correlated with cells that are at G0/G1 as indicated by PI and showed that 76% of ChroM1^{VE+} cells were in the small cell sized fraction. The expression of ChroM1^{VE+} was correlated with a cell size and a cell cycle stage. In addition, a two-color fluorescence analysis by FACS correlated the ChroM1^{VE+} expression with a cell cycle antigen. ChroM1^{VE+} was assessed by double staining of transcription factors and cyclins that are markers for cell cycle. A high level for c-Fos, lower levels for c-Jun, Ki-67 and cyclin D1 marked cells at G1 to S phase, and a low level for cyclin B1 that is expressed at S to G2/M stage was detected (FIG. 8). These results summarize the finding that the MSC were double stained for ChroM1, where the specific markers of cell cycle correlate with ChroM1^{VE+} cells that are at the G₁ stage and that are complementary to the PI staining for DNA content (FIG. 7B).

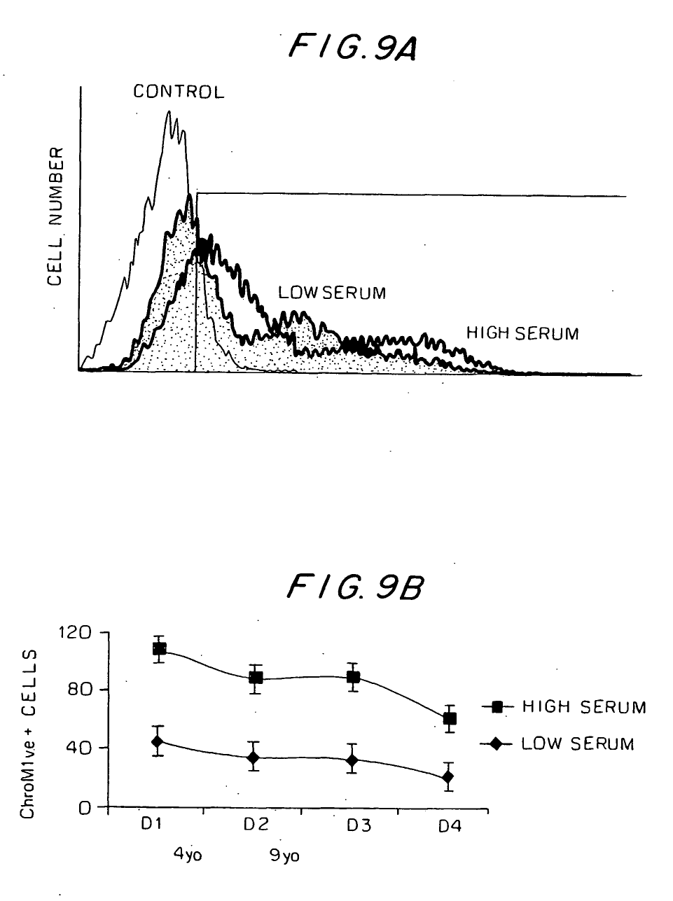

The ChroM1 expression was correlated with the MSC proliferation capacity in the presence of serum. Cells were cultured and maintained for one week in growth medium, and then were replaced with either media for growth conditions (10% FCS) or under low. serum (2% FCS) for an additional week. After the treatment, the cells were released and analyzed by FACS for ChroM1 expression. Under low serum conditions, lower cell count and a decrease in cell number of ChroM1^{VE+} expression were observed (FIG. 9A). MSC derived from four experiments, demonstrated a significant decrease of 30-45% compared with cells grown under regular growth conditions (FIG. 9B).

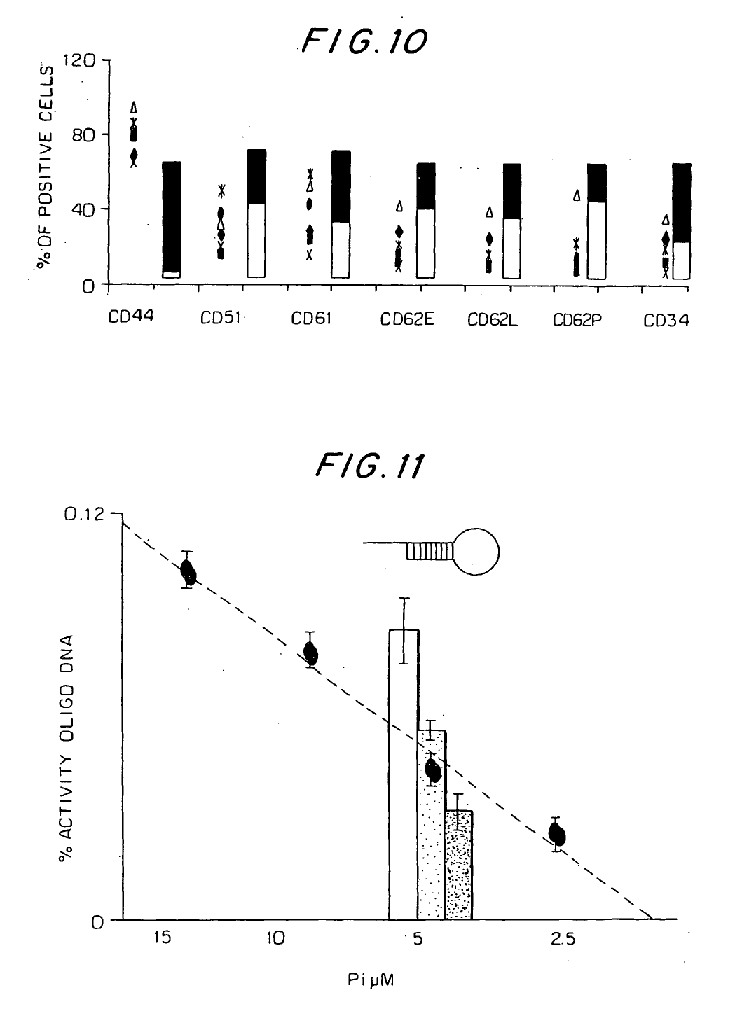

Flow cytometric analyses were used to quantify the co-expression of ChroM1 with a series of other surfaced markers (CD-44, integrins CD-51, CD-61, selections CD-62E, CD-62L, CD-62P and CD-34). In each experiment, the laboratory of the present inventors analyzed four subpopulations (immunonegative cells, ChroM1^{+VE} co-expressing one of the CD markers). The analysis was performed with MSC from 5 to 10 different donors (FIG. 10 and Table 8). The graph shown in FIG. 10 summarized the levels of expression for each specific antigen by different donors (shapes) and the respective percent of Chrom1^{+ve} cells that express either both antigens (dark bars) or ChroM1 only (white bars).

TABLE 8
FACS analysis of double staining for MSC

| | Double staining | | ChroM1 |
|---|---|---|---|
| Antigen | with ChroM1* | CD only | only |
| ChroM1 | | | 55 ± 18 |
| | | | (N = 10) |
| CD44 (N = 7) | 42 ± 18 | 52 ± 11 | 3 ± 4 |
| CD34 (N = 6) | 20 ± 8.7 | 2.25 ± 2 | 24.5 ± 17 |
| CD51 (N = 6) | 27 ± 14 | 3.5 ± 2 | 18 ± 9 |
| CD61 (N = 6) | 33 ± 17 | 7 ± 4.8 | 10 ± 6 |
| CD62E (N = 6) | 21 ± 12 | 1 ± 1 | 19 ± 16 |
| CD62L (N = 6) | 19 ± 11 | 1.2 ± 1 | 23 ± 17 |
| CD62P (N = 6) | 18.4 ± 15 | 2.2 ± 2 | 24 ± 12.7 |

*Data represent percent of positively stained cells from the total cell population expressed as mean 1 standard deviation. Number of donors checked appears in brackets.

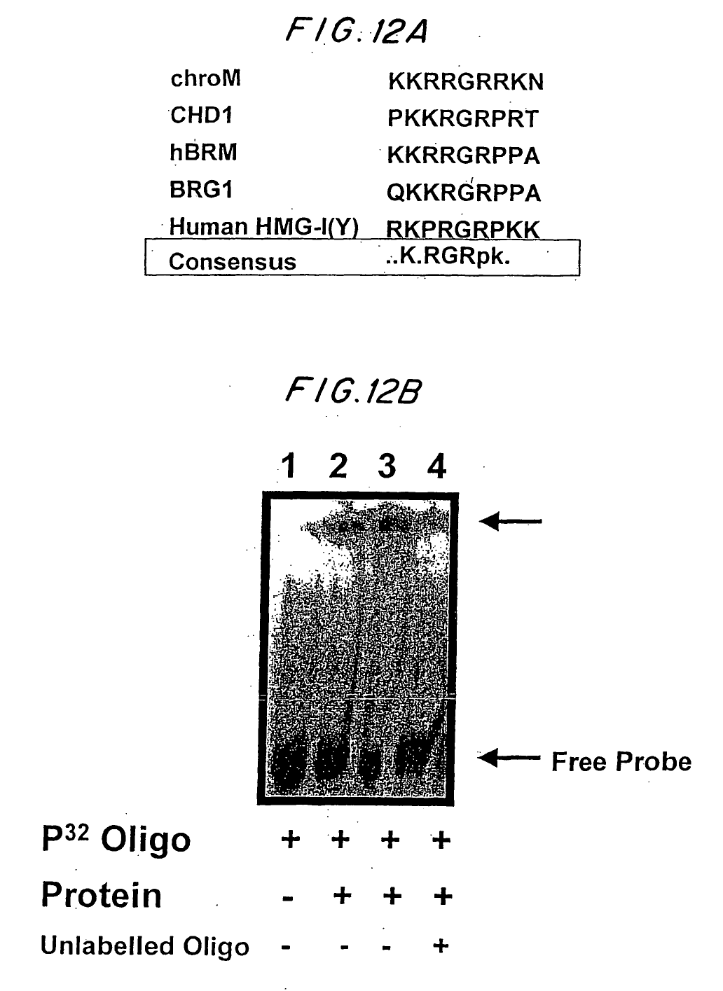

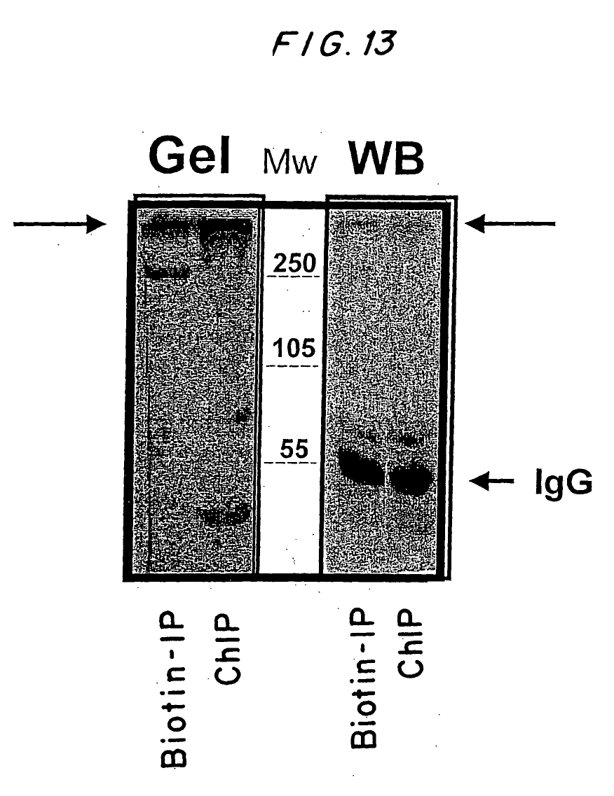

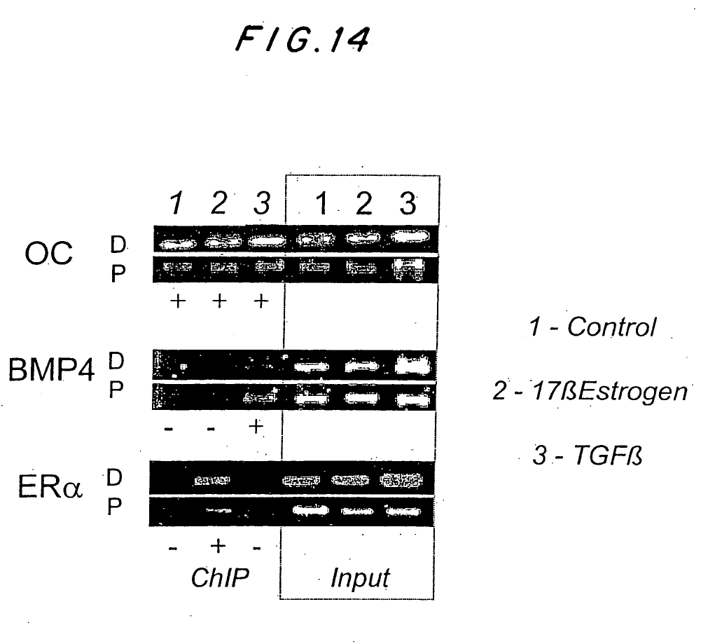

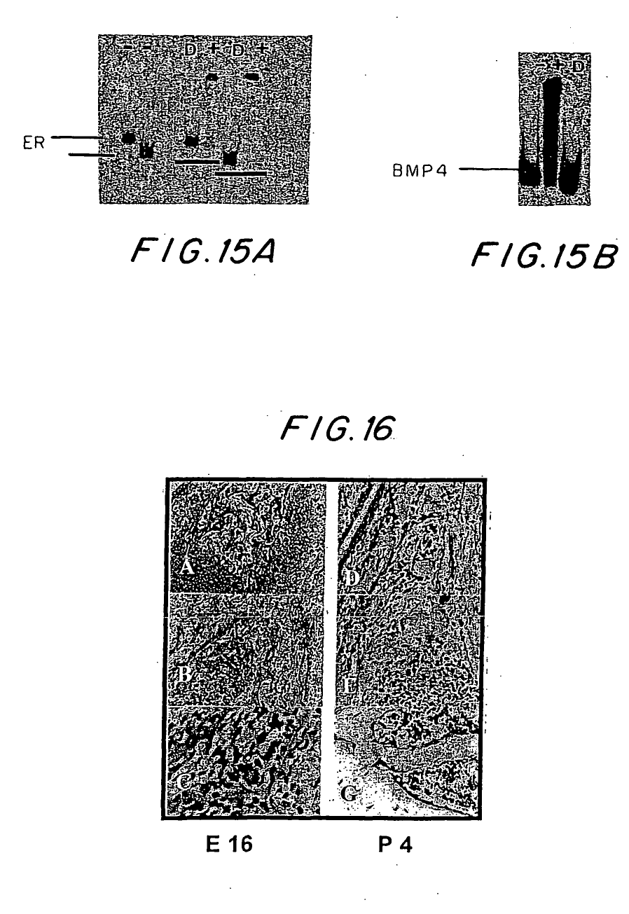

Functional domains proven for their activity at the N-terminus, an SNF2 with ATPase activity (FIG. 11), and at the C-terminus, a KR domain that functions as a DNA binding domain (DBD) (FIG. 12). The protein analysis employed immunoprecipitates (IP) (FIG. 13) from total cell extracts or biotinylated preparations of cell membrane, or after cross-linking of protein-nucleic acid, with formaldehyde (X-CHIP). This enabled the ability to follow the existence and dynamics of protein binding to chromatin. The IP of ChroM1 protein and the protein-DNA complexes were detected by Western blot and showed a protein of approximately 320 kDa (arrows, FIG. 13). This IP of the protein from total cell extract also showed a lower band of 250 KDa that co-immunoprecipitates with ChroM1 (FIG. 13). This band was analyzed by Maldi-mass spectroscopy and electrospray and identified as a non-skeletal myosin II. The electrospray results provide evidence for a complex between chromatin protein and a cytoskeletal component as was suggested by the bioinformatic analysis. The IP of ChroM1 from MSC extracts was used to quantify ATPase activity and was measured in the presence of stem-loop DNA. ATPase activity determined the Pi as 5 μM release per sample using a standard curve of inorganic phosphate (FIG. 11). Coupling the ATPase domain with the DNA binding domain is conserved in the SNF2 family members (FIG. 12) and is required for the proper activation of the DNA-dependent ATPase activity. To prove such an interaction, a recombinant protein (rP) that contains the KR region and is recognized as AT-rich binding site that bind to DNA was used. The KR region is functionally and structurally similar to the DNA binding domain of HMGI/Y proteins. The rP used for EMSA was hybridized with a ³²P-labeled primer rich in A/T sequence. In the presence of rP and the primer, gel mobility was visualized (FIG. 12B). Two protein concentrations (1 μg and 3 μg) were used that result in the increase in binding of the radioactive labeled primer. Addition of a 100-fold excess of the cold (unlabeled) primer abolished the binding of the radiolabeled primer (FIG. 12B). Further analysis to determine if ChroM1 has the availability to bind to specific promoters was performed. Using the ChIP assay, three promoters were identified: estrogen receptor (ERα), BMP4 and osteocalcin that were amplified by PCR. The PCR amplified from two regions: a proximal promoter (P) to the gene translation initiation and a distal promoter (D) region that is the 5′ flanking region for the promoter (FIGS. 14, 15, Table 9). The distal region of each promoter was chosen based on its A/T content, which was 65% to 75%. Estrogen receptor (ER) is highly expressed in proliferating osteoblasts; BMP4 gene from the TGFβ family regulates the first stages of bone matrix mineralization and osteocalcin serves as a marker for osteoblast differentiation. A base level for the expression of PCR amplified osteocalcin promoter (SEQ ID NO:34) could be identified but not for BMP4 or ERα promoters. MSC were treated with 17-β-estradiol (10⁻⁸M) or with TGFβ (5 ng/ml) for 24 hrs. in culture and then were analyzed by the ChIP. A positive signal for the ERα promoter following estrogen treatment and for BMP4 promoter following TGFβ treatment was detected (FIG. 14). The quality of DNA was analyzed also in the corresponding input chromatin fraction. This PCR fragment from the distal region was used further as a probe for EMSA with the recombinant protein (rp). Each labeled probe was analyzed when bound to the recombinant protein with a shift of the complex, the gel shift result with detection of higher band (FIG. 15). The binding was reduced in the presence of distamycin A, an antibiotic that blocks the binding to A/T rich regions. Distamycin A is an antibiotic, characterized by an oligopeptidic pyrrolocarbamoyl frame ending with an amidino moiety, which binds reversibly to the DNA minor groove with high selectivity for A/T-rich sequences and shows antiviral and anti-protozoal activity (Cozzi P, Mongelli N., 1998)

TABLE 9

| | | |
|---|---|---|
| | | Size of PCR |
| Promoter | Primers | product |

| | | | | |
|---|---|---|---|---|
| BMP4 | BMP dF | GCTAAAGGAGCACAATGCCT | (SEQ ID NO:20) | 145 bp |
| | BMP dR | CCCCAAAAGGAGGACAAAAT | (SEQ ID NO:21) | |
| BMP4 | BMP pF | TAGTACCTCCGCACGTGGTC | (SEQ ID NO:22) | 457 bp |
| | BMP pR | CTGCAGGCTCGAGATAGCTT | (SEQ ID NO:23) | |
| Osteocalcin | OC dF | ACCAGCCTACAGGCTCTTTTT | (SEQ ID NO:24) | 113 bp |
| | OC dR | AGAGCCAGACCCTGTCTCAA | (SEQ ID NO:25) | |
| Osteocalcin | OC pF | AGGCTGCCTTTGGTGACTC | (SEQ ID NO:26) | 497 bp |
| | OC pR | TTATACCCTCTGGGCTGTGC | (SEQ ID NO:27) | |
| Estrogen | Erα dF | CGCATGATATACTTCACCTATTTTT | (SEQ ID NO:28) | 204 bp |
| receptor α | Erα dR | TTGGGCTAGGATATGCAGAA | (SEQ ID NO:29) | |
| Estrogen | Erα pF | AACAGCCTCCTGTCTACCGA | (SEQ ID NO:30) | 110 bp |
| receptor α | Erα pR | CAGGAGAAAGGAGCATGGAC | (SEQ ID NO:31) | |

Expression of ChroM in Embryogenesis

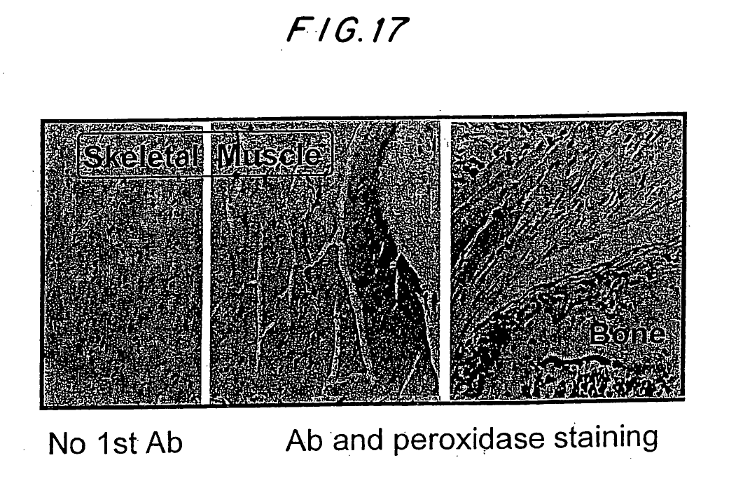

The presence of the ChroM1 protein was identified also during embryonic development. Immunostaining of sections from mouse embryo at 16.5 days resulted with staining in mesenchymal condensation during skeletal development and its appearance with development (FIG. 16). Positive staining was also detected in progenitors at the skeletal muscle (FIG. 17). These results suggest that the identification of progenitors give rise to two cell lineages (osteogenic and skeletal). The histological staining detects the mesenchymal progenitors on the developmental level.

Histopathology of Human Bone Tumors

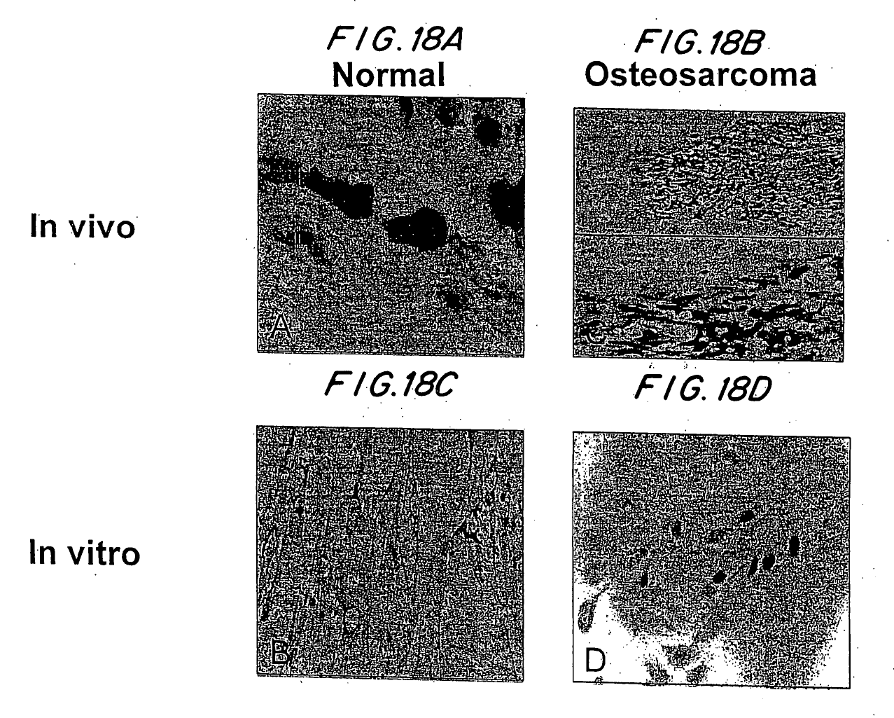

The ChroM1 expression in tissue sections of normal bone (FIG. 18A) or cultured MSC (FIG. 18B) was membranous or cytoplasmic. Immunostaining of biopsies of bone cancer osteogenic sarcoma (OS), the most frequent primary, highly malignant tumor cells, occur in poorly differentiated cells. Such cells produce the malignant state and appear mainly in children or young adults and are shown in FIG. 18C. The tumor cells of osteosarcoma were examined in biopsies and the cells lines were observed to express a different cellular pattern of the protein, where the protein is translocated to the nucleus (FIG. 18D).

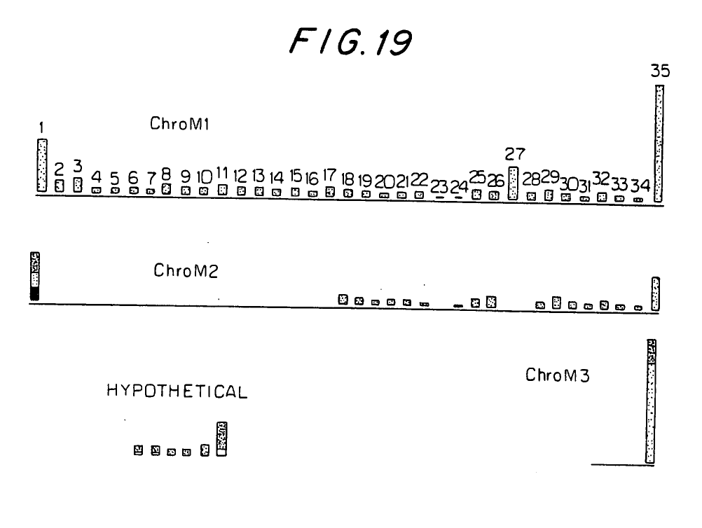

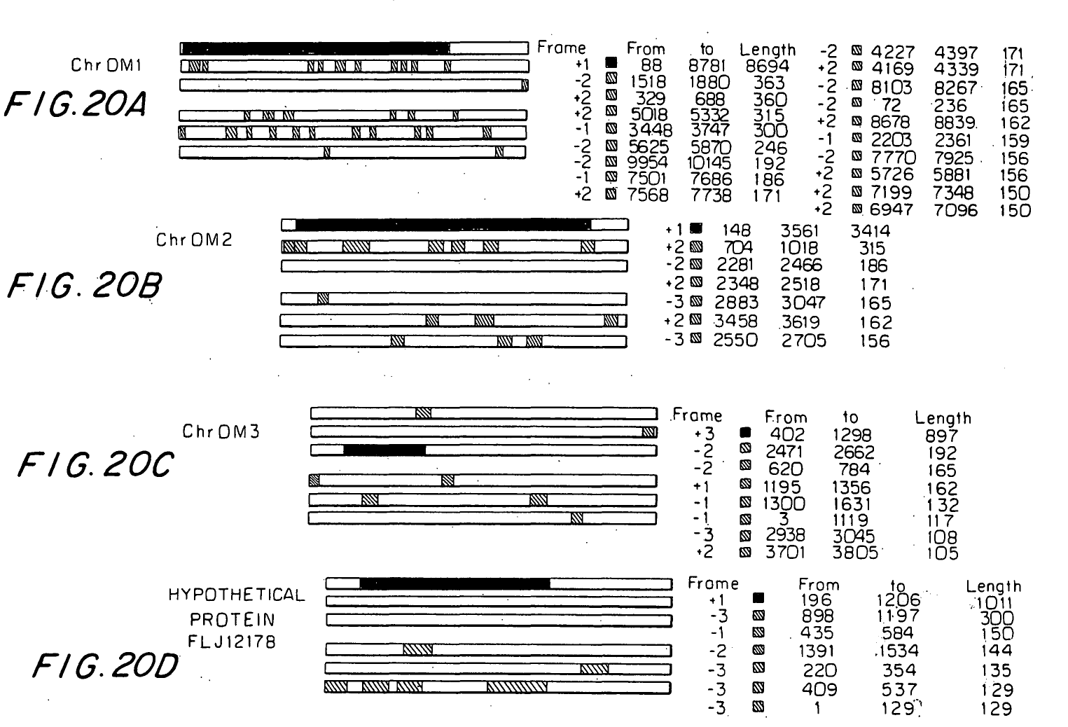

The cDNA of other alternative splice forms were cloned (FIGS. 19 and 20A-20D) and further computer predicted splice forms were then also verified by RT-PCR and the predicted transcripts were obtained (Table 10).

TABLE 10
Identified and predicted splice variants of ChroM

| | | | |
|---|---|---|---|
| Splice variant | Translation initiation | Unique feature | Protein domains |

GENE/PROTEIN-SCHEME

| | | | |
|---|---|---|---|
| ChrOM1 | + | 5′ of exon 1 | |
| ChrOM2 | + | 1) missing 5′ and 3′ of 1 and | SANT, KR-like |
| | | translation of 36 bp only | |
| | | 2) missing exon 27 | |
| ChrOM3 | + | 5′ of exon 35: 5′ UTR+ translation | TM |
| Hypothetical protein | + | 5′ UTR of exon 7: | SNF2 |
| FLJ12178 | | 3′ UTR of exon 12 | |

EST AND PARTIAL CDNA VERIFIED BY RT-PCR *

| | | | |
|---|---|---|---|
| ChrOM4 | * | Extra 3 bp 5′ exon 28 | |
| Kiaa0308 | + | Missing 3′ exon 29 | |
| BE882430 (ChroM5) | + | Extra exon 1a | Hook, b-catenine binding |
| W88543 | * | Extra exon 26a | AAA TRIP 13 like |
| Spleen/fetal liver | | | |
| ChroM | * | Extra exon 15p | Signal peptide |
| Pred1 | | | |
| ChroM | * | Extra exon 23p | Signal peptide |
| Pred2 | | | |

1Not verified by RT-PCR from MSC W99387 from fetal heart,
2Not expressed in RT-PCR of MSC AA009999 fetal spleen

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### References

Artavanis-Tsakonas S, Matsuno K, Fortini M E. Notch signaling. Science. 14;268(5208):225-32. Review. (April 1995)
Ausubel et al (Eds), Current Protocols in Molecular Biology, John Wiley & Sons, Inc. (New York) (1987-1999)
Baykov A A, Evtushenko O A, Avaeva S M :A Malachite Green Procedure for Orthophosphate Determination and its Use in Alkaline Phosphatase-Based Enzyme Immunoassay, Analytical Biochemistry 171:266-270 (1988)
Better et al, “Escherichia coli secretion of an active chimeric antibody fragment”, Science 240d(4855):1041-1043 (1988)
Bourachot B, Yaniv M, Muchardt C. The activity of mammalian brm/SNF2alpha is dependent on a high-mobility-group protein I/Y-like DNA binding domain. Mol Cell Biol. 19(6):3931-9 (1999).
Chen H, Lin R J, Xie W, Wilpitz D, Evans R M. Regulation of hormone-induced histone hyperacetylation and gene activation via acetylation of an acetylase. Cell 98:675-686 (1999)
Colligan et al (eds.), Current Protocols in Immunology, John Wiley & Sons, Inc. (New York) (1992-2000)
Cozzi P, Mongelli N. Cytotoxics derived from distamycin A and congeners. Curr Pharm Des 1998;4(3):181-201
Daubresse G, Deuring R, Moore L, Papoulas O, Zakrajsek I, Waldrip W R, Scott M P, Kennison J A, Tamkun J W. The Drosophila kismet gene is related to chromatin-remodeling factors and is required for both segmentation and segment identity. Development, 126(6):1175-87 (1999).
Dilworth F J, Chambon P. Nuclear receptors coordinate the activities of chromatin remodeling complexes and coactivators to facilitate initiation of transcription. Oncogene, 20(24):3047-54 (2001)
Elfring L K, Daniel C, Papoulas O, Deuring R, Sarte M, Moseley S, Beek S J, Waldrip W R, Daubresse G, DePace A, Kennison J A, Tamkun J W. Genetic analysis of brahma: the Drosophila homolog of the yeast chromatin remodeling factor SWI2/SNF2. Genetics, 148(1):251-65 (1998)
Fry C J, Peterson C L. Chromatin remodeling enzymes: who's on first? Curr Biol, 11(5):R185-97 (2001)
Fryer C J, Archer T K. Chromatin remodelling by the glucocorticoid receptor requires the BRG1 complex. Nature, 7:393(6680):88-91 (1998).
Goodship A E and Cooke P. Bicompatibility of tendon and ligament prostheses. Critical Reviews in Biocompatibility 2(4):303-334 (1986)
Gottardi C J, Arpin M, Fanning A S, Louvard D. The junction-associated protein, zonula occludens-1, localizes to the nucleus before the maturation and during the remodeling of cell-cell contacts. Proc Natl Acad Sci U S A. 1996 1;93(20):10779-84 (October 1996)
Harlow et al, Antibodies: A Laboratory Manual, Cold Spring Harbor Laboratory (Cold Spring Harbor, N.Y.) (1988)
Ichinose H, Garnier J M, Chambon P, Losson R. Ligand-dependent interaction between the estrogen receptor and the human homologues of SWI2/SNF2. Gene, 188(1):95-100 (1997)
Jenkins B D, Pullen C B, Darimont B D. Novel glucocorticoid receptor coactivator effector mechanisms. Trends Endocrinol Metab, 12(3):122-6 (2001)
Kohler et al, “Continuous cultures of fused cells secreting antibody of predefined specificity”, Nature 256(5517):495-497 (1975)
Lian J B, Stein G S, Stein J L, van Wijnen A J. Osteocalcin gene promoter: unlocking the secrets for regulation of osteoblast growth and differentiation. J Cell Biochem Suppl. 1998;30-31:62-72. Review
Liu et al, “Chimeric mouse-human IgG1 antibody that can mediate lysis of cancer cells”, Proc Natl Acad Sci USA. 1987 May;84(10):3439-3443 (1987)
Manolagas S C, Weinstein R S. New developments in the pathogenesis and treatment of steroid-induced osteoporosis. J Bone Miner Res,14(7):1061-6 (1999)
Meinkoth et al, “Hybridization of nucleic acids immobilized on solid supports”, Anal Biochem 138:267-284 (1984)
Morrison et al, “Chimeric human antibody molecules: mouse antigen-binding domains with human constant region domains”, Proc Natl Acad Sci USA 81(21):6851-6855 (1984)
Muchardt C, Yaniv M. A human homologue of Saccharomyces cerevisiae SNF2/SWI2 and Drosophila brm genes potentiates transcriptional activation by the glucocorticoid receptor. EMBO J., 12(11):4279-90 (1993)
Muthuswami R, Mesner L D, Wang D, Hill D A, Imbalzano A N, Hockensmith J W: “A eukaryotic SWI2/SNF2 domain, an exquisite detector of double-stranded to single-stranded DNA transition elements”. J Biol Chem, 17:275(11):7648-55 (2000)
Nakamura, T., Blechman, J., Tada, S., Rozovskaia, T., Itoyama, T., Bullrich, F., Mazo, A., Croce, C. M., Geiger, B. and Canaani, E. huASH1 protein, a putative transcription factor encoded by a human homologue of the Drosophila ash1 gene, localizes to both nuclei and cell-cell tight junctions Proc. Natl. Acad. Sci. U.S.A., 97 (13), 7284-7289 (2000)
Neuberger et al, “A hapten-specific chimaeric IgE antibody with human physiological effector function”, Nature 314(6008):268-270 (1985)
Orlando V. Mapping chromosomal proteins in vivo by formaldehyde-crosslinked-chromatin immunoprecipitation. Trends Biochem Sci, 25(3):99-104 (2000)
Ouyang P. Antibodies differentiate desmosome-form and nucleus-form pinin: evidence that pinin is a moonlighting protein with dual location at the desmosome and within the nucleus. Biochem Biophys Res Commun. September 16;263(1):192-200 (1999)
Queen et al., Proc. Natl. Acad. Sci. USA, 86:10029-10033 (1989)
Shur I, Kuznetsov S, DeRubeis A R, Gehron-Robey P, Benayahu D, Differential genes expression by human marrow stromal cloned cells. Chemistry & Biology of Mineralized Tissues {Goldberg M, Boskey A and Robinsonb C., Editors}. Publisher, American Academy of Orthopaedic Surgeons, pp 69-74 (2000)
Spelsberg T C, Subramaniam M, Riggs B L, Khosla S.The actions and interactions of sex steroids and growth factors/cytokines on the skeleton. Mol Endocrinol, 13(6):819-28 (1999)
Therrien M, Morrison D K, Wong A M, Rubin G M. A genetic screen for modifiers of a kinase suppressor of Ras-dependent rough eye phenotype in Drosophila. Genetics, 156(3):1231-42 (2000)
van den Wijngaard A, Mulder W R, Dijkema R, Boersma C J, Mosselman S, van Zoelen E J, Olijve W.Antiestrogens specifically up-regulate bone morphogenetic protein-4 promoter activity in human osteoblastic cells. Mol Endocrinol. 2000 May;14(5):623-33.
Yang L, Guerrero J, Hong H, DeFranco D B, Stallcup M R. Interaction of the tau2 transcriptional activation domain of glucocorticoid receptor with a novel steroid receptor coactivator, Hic-5, which localizes to both focal adhesions and the nuclear matrix. Mol Biol Cell. June; 11(6):2007-18 (2000)

1. An isolated polypeptide having the activity of a chromatin remodeling protein, comprising:
(a) the amino acid sequence of SEQ ID NO:2;
(b) a fragment of (a);
(c) an amino acid sequence having at least 95% sequence identity to the amino acid sequence of SEQ ID NO:2; or
(d) a fragment of (c),
wherein said fragment of (a) or of (c) has the activity of the chromatin remodeling protein of SEQ ID NO:2.

2. The isolated polypeptide of claim 1, which comprises the amino acid sequence of SEQ ID NO:2.

3. An isolated naturally occurring variant of the polypeptide of claim 2, comprising the amino acid sequence of SEQ ID NO:4.

4. A molecule comprising the antigen binding portion of an antibody against the variant polypeptide of claim 3.

5. An isolated naturally occurring variant of the polypeptide of claim 2, comprising the amino acid sequence of SEQ ID NO:6.

6. A molecule comprising the antigen binding portion of an antibody against the variant polypeptide of claim 5.

7. An isolated naturally occurring variant of the polypeptide of claim 2, comprising the amino acid sequence of SEQ ID NO:8.

8. A molecule comprising the antigen binding portion of an antibody against the variant polypeptide of claim 7.

9. The isolated polypeptide of claim 1, which comprises a fragment of (a).

10. The isolated polypeptide of claim 1, which comprises an amino acid sequence having at least 95% sequence identity to the amino acid sequence of SEQ ID NO:2.

11. The isolated polypeptide of claim 1, which comprises a fragment of (c).

12. A composition, comprising the polypeptide of claim 1 and an excipient, diluent, carrier or auxiliary agent.

13. A molecule comprising the antigen binding portion of an antibody against the polypeptide of claim 1.

14. The molecule of claim 13, which is a polyclonal antibody.

15. The molecule of claim 13, which is a monoclonal antibody.

16. A method for recovering human stromal progenitor cells from a cell mixture derived from human bone marrow, comprising:
contacting the cell mixture with a molecule of claim 13 which is an antibody that selectively binds to an antigen on human osteogenic and muscle progenitor cells; and recovering antibody-bound cells from the cell mixture.

17. The method of claim 16, wherein the antigen on human osteogenic progenitor cells which is selectively bound with the molecule is a polypeptide comprising the amino acid sequence of SEQ ID NO:2.

18. The method of claim 16, wherein the human stromal progenitor cells are isolated culture-expanded human osteogenic progenitor cells.

19. A method for separating a cell population containing human osteogenic progenitor cells from a cell mixture derived from human bone marrow, comprising:
contacting the cell mixture with a molecule of claim 13 which is an antibody that selectively binds to an antigen on human osteogenic progenitor cells; and
separating antibody-bound cells from the cell mixture.

20. The method of claim 19, wherein the antigen on human osteogenic progenitor cells which is selectively bound with the molecule is a polypeptide comprising the amino acid sequence of SEQ ID NO:2.

21. The method of claim 19 wherein the antibody-bound cells are labeled with a fluorescent tag and the fluorescent tagged, antibody-bound cells are separated by fluorescence activated cell sorting.

22. The method of claim 19, wherein the antibody is a polyclonal antibody which is immobilized on a solid support prior to the step of contacting.

23. The method of claim 22, wherein the solid support is a column or magnetic beads.

24. The method of claim 19, wherein the human osteogenic progenitor cells are isolated, culture-expanded human osteogenic progenitor cells.

25. The method of claim 19, further comprising:
contacting the separated antibody-bound cells with a second antibody which selectively binds to a second antigen on a subpopulation of human osteogenic progenitor cells; and
separating the second antibody-bound cells as a subpopulation from the population of human osteogenic progenitor cells.

26. The method of claim 25, wherein the subpopulation of second antibody-bound cells is an isolated, culture-expanded subpopulation of human osteogenic progenitor cells.

27. An isolated population of cells enriched for human osteogenic progenitor cells, wherein greater than 30% of said population of cells are positive for the presence of the polypeptide of SEQ ID NO:2 and can differentiate into osteogenic cells and muscle cells.

28. The isolated population of cells of claim 27, wherein greater than 40% of said population of cells are positive for the presence of the polypeptide of SEQ ID NO:2 and can differentiate into osteogenic cells and muscle cells.

29. The isolated population of cells of claim 27, wherein greater than 50% of said population of cells are positive for the presence of the polypeptide of SEQ ID NO:2 and can differentiate into osteogenic cells and muscle cells.

30. The isolated population of cells of claim 27, wherein greater than 60% of said population of cells are positive for the presence of the polypeptide of SEQ ID NO:2 and can differentiate into osteogenic cells and muscle cells.

31. A composition comprising a physiologically acceptable medium and the isolated population of cells of claim 27.

32. A method of treating bone or muscle tissue damage, comprising administering to a patient in need thereof the isolated population of cells enriched for human osteogenic progenitor cells of claim 27 which are culture expanded and which are autologous to the patient.

33. A method of generating bone or muscle tissue, comprising:
seeding a matrix or scaffold for generating bone or muscle tissue with the isolated population of cells enriched for human osteogenic progenitor cells of claim 27 that have been culture expanded,
inducing differentiation of the culture expanded population of cells to generate bone or muscle tissue using the matrix or scaffold as support.

34. A method for identifying osteosarcoma cells in a tissue sample, comprising:
contacting a tissue sample containing osteogenic progenitor cells with a molecule of claim 13; and
detecting by immunohistochemistry the presence of the molecule in the nuclei of cells from tissue biopsies to identify osteosarcoma cells.

35. A method for evaluating the effectiveness of a treatment for osteogenic progenitor sarcoma, comprising:
contacting a tissue sample containing osteogenic progenitor cells with a molecule of claim 13; and
detecting by the immunohistochemistry the presence of the molecule in nuclei of cells in the tissue sample to identify osteosarcoma progenitor cells and to evaluate the effectiveness of the treatment.

36. A method of screening for and identifying an enhancer or inhibitor compound that affect expression of the chromatin remodeling protein ChroM1, comprising:
incubating a human cell, which expresses the ChroM1 polypeptide of SEQ ID NO:2, in the presence or absence of a potential enhancer or inhibitor compound that affects expression of ChroM1;
determining the level of expression of the ChroM1 polypeptide in the presence of the potential enhancer or inhibitor compound relative to the level of expression of the ChroM1 polypeptide in the absence of the potential enhancer or inhibitor; and
identifying as an enhancer compound any potential enhancer or inhibitor compound for which said determining step determines that the level of expression of the ChroM1 polypeptide in the presence of the potential enhancer compound is substantially more than that in the absence of the potential enhancer compound and identifying as an inhibitor compound any potential enhancer or inhibitor compound for which said determining step determines that the level of expression of ChroM1 polypeptide in the presence of the potential inhibitor compound is substantially less than that in the absence of the potential inhibitor compound.

37. The method of claim 36, further comprising a step of isolating the enhancer or inhibitor identified in said identifying step.

38. A method for treating osteoporosis, comprising administering an enhancer compound identified in the method of claim 36 to a patient in need thereof.

39. A method for treating osteopetrosis or osteosarcoma, comprising administering an inhibitor compound identified in the method of claim 36 to a patient in need thereof.

40. A method of identifying A/T-rich promoter regions of genes involved in modulation of osteoblast differentiation and capable of binding to the DNA binding domain of the ChroM1 chromatin remodeling protein, comprising:
contacting fragments of genomic DNA with a peptide containing a DNA binding domain comprising the amino acid sequence of residues 2429-2437 of SEQ ID NO:2;
identifying a fragment of genomic DNA bound by said peptide;
determining the nucleotide sequence of the promoter region on said fragment bound by said peptide to identify an A/T-rich promoter region of a gene involved in modulation of osteoblast differentiation.

41. The method of claim 40, wherein said peptide containing a DNA binding domain comprises the amino acid sequence of residues 2333 to 2480 of SEQ ID NO:2.

42. A method of screening for and identifying a compound which stimulate differentiation of osteogenic progenitor cells, comprising:
incubating human cells, which express the ChroM1 chromatin remodeling protein of SEQ ID NO:2, with a potential stimulator compound;
adding formaldehyde to the incubated human cells to crosslink proteins to DNA in chromatin by in vivo fixation;
sonicating the crosslinked chromatin to solubilize the crosslinked chromatin;
immunoprecipitating the solubilized crosslinked chromatin with antibodies specific for the ChroM1 protein of SEQ ID NO:2 to form immunocomplexes;
recovering DNA from the immunocomplexes;
incubating under amplification conditions the recovered DNA with oligonucleotide primers capable of amplifying an A/T-rich promoter region of a gene involved in control of osteoblast cell differentiation if present in the recovered DNA; and
detecting an amplification product and identifying as a stimulator compound any potential stimulator compound for which an amplification product corresponding to said AT-rich promoter region is detected step.

43. The method of claim 42, wherein the A/T-rich promoter region is selected from the group consisting of the human estrogen receptor (ER) promoter and the human bone morphogenic protein (BMP) promoter.

44. The method of claim 43, wherein the human estrogen receptor promoter is the human estrogen receptor α (ERα) promoter.

45. The method of claim 43, wherein the human bone morphogenic protein promoter is the human bone morphogenic protein-4 (BMP-4) promoter.

46. An isolated nucleic acid molecule, comprising a nucleotide sequence encoding the polypeptide of claim 1.

47. The isolated nucleic acid molecule of claim 46, wherein the polypeptide comprises the amino acid sequence of SEQ ID NO:2.

48. The isolated nucleic acid molecule of claim 46, wherein said nucleotide sequence encoding said polypeptide comprises nucleotides 88 to 8781 of SEQ ID NO:1.

49. The isolated nucleic acid molecule of claim 46, wherein the polypeptide comprises a fragment of (a).

50. The isolated nucleic acid molecule of claim 46, wherein the polypeptide comprises an amino acid sequence having at least 95% sequence identity to the amino acid sequence of SEQ ID NO:2.

51. The isolated nucleic acid molecule of claim 46, wherein the polypeptide comprises a fragment of (c).

52. A vector, comprising the nucleic acid of claim 46.

53. A host cell transformed with the nucleic acid of claim 46.

54. An isolated nucleic acid molecule which hybridizes to the nucleotide sequence of the nucleic acid molecule of claim 46 under high stringency conditions.

55. A process for preparing a polypeptide comprising the amino acid sequence of SEQ ID NO:2 or a fragment thereof, comprising:
recombinantly expressing the polypeptide from the nucleic acid molecule of claim 46; and
recovering the expressed polypeptide to prepare the polypeptide comprising the amino acid sequence of SEQ ID NO:2 or a fragment thereof.

56. An antisense oligonucleotide complementary to a messenger RNA comprising nucleotides 88 to 8781 of SEQ ID NO:1 and encoding a polypeptide having the activity of a chromatin remodeling protein, wherein said oligonucleotide inhibits the production of said polypeptide.

57. A method for treating osteopetrosis or osteosarcoma, comprising administering the antisense oligonucleotide of claim 56 to a patient in need thereof.

58. An isolated nucleic acid molecule, comprising a nucleotide sequence encoding the naturally occurring variant polypeptide of claim 3.

59. The isolated nucleic acid molecule of claim 58, wherein said nucleotide sequence comprises nucleotides 148 to 3558 of SEQ ID NO:3.

60. An isolated nucleic acid molecule, comprising a nucleotide sequence encoding the naturally occurring variant polypeptide of claim 5.

61. The isolated nucleic acid molecule of claim 60, wherein said nucleotide sequence comprises nucleotides 402 to 1298 of SEQ ID NO:5.

62. An isolated nucleic acid molecule, comprising a nucleotide sequence encoding the naturally occurring variant polypeptide of claim 7.

63. The isolated nucleic acid molecule of claim 62, wherein said nucleotide sequence comprises nucleotides 196 to 1206 of SEQ ID NO:7.

64. An isolated DNA molecule, comprising a nucleotide sequence encoding the amino acid sequence of SEQ ID NO:2, wherein said nucleotide sequence consists of the sequence of a fragment of human genomic DNA.
